(19) United States
(12) Patent Application Publication
(10) Pub. No.: US 2005/0084875 A1
(43) Pub. Date: Apr. 21, 2005

(54) Molecular markers for the diagnosis of Alzheimer's disease

(75) Inventors: Paul D. Coleman, Rochester, NY (US); Nienwen Chow, Rochester, NY (US); Christopher Cox, Pittsford, NY (US)

Correspondence Address: FISH & RICHARDSON PC, 225 FRANKLIN ST, BOSTON, MA, 02110, US

(73) Assignee: University of Rochester

(21) Appl. No.: 10/821,653

(22) Filed: Apr. 9, 2004

Related U.S. Application Data
(63) Continuation-in-part of application No. 09/770,534, filed on Jan. 25, 2001, now abandoned.
Application No. 09/770,534 is a continuation of application No. 09/178,170, filed on Oct. 23, 1998, now abandoned.
(60) Provisional application No. 60/063,274, filed on Oct. 24, 1997.

(51) Int. Cl. C12Q 1/68

(52) U.S. Cl. 435/6

(57) ABSTRACT

The present invention provides methods for profiling mRNA production in single cells, in particular, during disease stages. The invention also provides a method for monitoring gene expression in a single cell which has been isolated from tissue. The methods of the present invention may be used to diagnose or monitor the progression of disease.

The present application claims the benefit of U.S. Provisional Patent Application Ser. No. 60/063,274, filed Oct. 24, 1997.

The subject matter of this application was made with support from the United States Government under Grant Nos. AG09016, RO1 AG01121 and ADC AG08665 from the National Institutes of Health. The United States Government may retain certain rights.

### FIELD OF THE INVENTION

The present invention relates to molecular markers which are useful for determining susceptibility to the development of Alzheimer's disease in a patient. The invention also provides methods for determining whether a patient is susceptible to the development of Alzheimer's disease.

### BACKGROUND OF THE INVENTION

The original paper in which Alois Alzheimer presented the case of a 51 year old woman with dementing illness described two lesions in the brain of this patient: dense intracellular bundles of abnormal fibrils and “miliary foci” (Alzheimer, “Uber Eine Eigenartife Erkrankung der Hinrinde,” Allg. Z. Psychiat. 64:146-48 (1907)). The miliary foci are now known as senile plaques (“SP”) and the abnormal fibrils as neurofibrillary tangles (“NFT”). Although these classic lesions have been known for 90 years, their role in altering brain function to produce the cognitive deficits of Alzheimer's disease (“AD”) remains uncertain. Furthermore, the relationship of these classic lesions to most of the many alternations in gene expression described in the AD brain are not yet knowvn.

Although, the relationship between SP or NFT and cognition remains unclear, a great deal has been learned about the composition of these lesions. The characterization of amyloid by Glenner, G. G. et al., “Alzheimer's disease: Initial Report of the Purification and Characterization of a Novel Cerebrovascular Amyloid Protein.” Biochem. Biophys. Res. Comm., 120(3):885-90 (1984) led to an explosion of studies showing alternate splicing of APP message (e.g. Palmert, M. R. et al., “Amyloid Protein Precursor Messenger RNAs: Differential Expression in Alzheimer's Disease,” Science, 241:1080-4 (1988); Tanzi. R. E. et al., “Protease Inhibitor Domain Encoded By an Amyloid Protein Precursor mRNA Associated With Alzheimer's Disease.” Nature, 331:528-30 (1988); Hartmann, T. et al., “Alzheimer's Disease Beta A4 Protein Release and Amyloid Precursor Protein Sorting are Regulated by Alternative Splicing,” J. Bio. Chem., 27(22):13208-14 (1996)), variations in postranslational processing (e.g. Citron, M. et al., “Evidence that the 42- and 40-Amino Acid Forms of Amyloid Beta Protein are Generated from the Beta-Amyloid Precursor Protein by Different Protease Activities,” Proc. Nat'l Acad. Sci. U.S.A., 93(23):13170-5 (1996); Tischer, E. et al., “Beta-Amyloid Precursor Protein. Location of Transmembrane Domain and Specificity of Gamma-Secretase Cleavage,” J. Biol. Chem., 271:21914-9 (1996); Refolo, L. M. et al., “Evidence that Secretase Cleavage of Cell Surface Alzheimer Amyloid Precursor Occurs After Normal Endocytic Internalization,” J. Neurosci. Res., 40:694-706 (1995); Anderson, J. P. et al., “An Alternative Secretase Cleavage Produces Soluble Alzheimer Amyloid Precursor Protein Containing a Potentially Amyloidogenic Sequence,” J. Neurochem., 59:2328-31 (1992)) and aggregation of Aβ (e.g. Podlisny, M. B. et al., “Aggregation of Secreted Amyloid Beta-Protein Into Sodium Dodecyl Sulfate-Stable Oligomers in Cell Culture,” J. Biol. Chem., 270:9564-70 (1995); Pike, C. J. et al., “Structure-Activity Analyses of Beta-Amyloid Peptides: Contributions of the Beta 25-35 Region to Aggregation and Neurotoxicity,” J. Neurochem., 64:253-65 (1995); Giordano, T. “Similarities Between Beta Amyloid Peptides 1-40 and 40-1: Effects on Aggregation, Toxicity in vitro, and Injection in Young and Aged Rats,” Exp. Neurol., 125:175-82 (1994); See Forloni, G., “Neurotoxicity of Beta-Amyloid and Prion Peptides,” Cur. Opin. Neurol., 9(6):492-500 (1996), for review). Other studies have examined the neurotoxicitv of Aβ both in vitro and in vivo (e.g. Lorenzo, A. et al., “Beta-Amyloid Neurotoxicity Requires Fibril Formation and is Inhibited By Congo Red,” Proc. Natl. Acad. Sci. U.S.A., 91(25):12243-7 (1994); Podlisny, M. B. et al., “Microinjection of Synthetic Amyloid Beta-Protein in Monkey Cerebral Cortex Fails to Produce Acute Neurotoxicity,” Am. J. Path., 142(1):17-24 (1993); Stein-Behrens, B. et al., “Failure of Beta-Amyloid Protein Fragment 25-35 to Cause Hippocampal Damage in the Rat,” Neurobiol. Aging, 13(5):577-9 (1992); Kowall, N. W. et al., “In vivo Neurotoxicity of Beta-Amyloid [Beta(1-40)] and the Beta(25-35) Fragment,” Neurbiol. Aging, 13:537-42 (1992); Yankner, B. A. et al., “Neurotoxicity of a Fragment of the Amyloid Precursor Associated with Alzheimer's Disease.” Science, 245:417-20 (1989)) with uncertain results. Some studies have explored the mechanism(s) by which Aβ may contribute to other pathologies of AD (e.g. Mattson, M. P. et al., “Different Amyloidogenic Peptides Share a Similar Mechanism of Neurotoxicity Involving Reactive Oxygen Species and Calcium,” Brain Res., 676:219-24 (1995); Paradis, E. et al., “Amyloid Beta Peptide of Alzheimer's Disease Downregulates Bcl-2 and Upregulates Bax Expression in Human Neurons,” J. Neurosci., 16(23):7533-9 (1996); Greenberg, S. M. et al., “Secreted Beta-Amyloid Precursor Protein Stimulates Mitogen-Activated Protein Kinase and Enhances Tau Phosphorylation,” Proc. Nat'l Acad. Sci. U.S.A., 91(15):7104-8 (1994); Greenberg, S. M. et al., “Amino-Terminal Region of the Beta-Amyloid Precursor Protein Activates Mitogen-Activated Protein Kinase,” Neurosci. Let., 198(1):52-6 (1995)), while still other studies have created transfected cells or transgenic mouse models overexpressing Aβ in an attempt to clarify the pathophysiology of this molecule (e.g. Masliah, E. et al., “Comparison of Neurodegenerative Pathology in Transgenic Mice Overexpressing V717F Beta-Amyloid Precursor Protein and Alzheimer's Disease,” J. Neurosci., 16:5795-811 (1996); Oster-Granite, M. L. et al., “Age-Dependent Neuronal and Synaptic Degeneration in Mice Transgenic for the C Terminus of the Amyloid Precursor Protein,” J. Neurosci., 16:6732-41 (1996); Hsiao, K. K. et al., “Age-Related CNS Disorder and Early Death in Transgenic FVB/N Mice Overexpressing Alzheimer Amyloid Precursor Proteins,” Neuron 15:1203-1218 (1995); Games, D. et al., “Alzheimer-Type Neuropathology in Transgenic Mice Overexpressing V717F Beta-Amyloid Precursor Protein,” Nature, 373(6514):523-7 (1995); Kammesheidt, A. et al., “Deposition of Beta/A4 Immunoreactivity and Neuronal Pathology in Transgenic Mice Expressing the Carboxyl-Terminal Fragment of the Alzheimer Amyloid Precursor in the Brain,” Proc. Nat'l Acad. Sci. U.S.A., 89:10857-61 (1992)). In spite of the tremendous advances in the understanding of APP and its Aβ product, the role that these may play in the pathological cascade of AD remains unclear (see earlier review by Yankner, B. et al., “β Amyloid and the Pathogenesis of AD,” New Engl. J. Med., 325:1849-1857 (1991)).

As is the case for Aβ, the bottom line for NFT is that although a great deal is known about the composition of NFT, there is insufficient understanding of the pathological cascade that leads to the formation of NFT and the mechanism(s) by which NFT may affect cognition. Since the intracellular NFT are more germane to this proposal than the extracellular SP, more attention will be devoted to reviewing the NFT in AD. The major component of the NFT and the constituent paired helical filaments (PHF) has been presumed to be the abnormally phosphorylated microtubule-associated protein, tau (e.g. Grundke-lqbal, I. et al., “Abnormal Phosphorylation of the Microtubule-Associated Protein Tau in Alzheimer Cytoskeletal Pathology,” Proc. Natl. Acad. Sci. U.S.A., 83:4913-4917 (1986); Goedert, M. et al., “Tau Proteins of Alzheimer Paired Helical Filaments: Abnormal Phosphorylation of All Six Brain Isoforms,” Neuron, 8:159-168 (1992a)). However, the role of tau phosphorylation in the formation of NFT may be questioned (e.g. Matsuo, E. S. et al., “Biopsy-Derived Adult Human Brain Tau is Phosphorylated at Many of the Same Sites as Alzheimer's Disease Paired Helical Filament Tau,” Neuron, 13:989-1002 (1994); but see Song, J. et al., “Low Initial Tau Phosphorylation in Human Brain Biopsy Samples,” Neurobiology of Aging, 18(5) (1997)).

The NFT are filamentous cytoplasmic inclusions that occupy the cell to varying extent. The principal structural components of NFT's are paired helical filaments (PHFs) (Kidd, M. “Paired Hilical Filaments in Electron Microscopy of Alzheimer's Disease,” Nature, 197:192-193 (1963)), whose major component is the microtubule-associated protein, tau, which has been altered from normal in ways that are still in dispute. There is little doubt, however, that conformational and phosphorylation changes enter the picture (e.g. Grundke-lqbal, I. et al., “Abnormal Phosphorylation of the Microtubule-Associated Protein Tau in Alzheimer Cytoskeletal Pathology,” Proc. Natl. Acd. Sci. U.S.A., 83:4913-4917 (1986); Goedert, M. et al., “Tau Proteins of Alzheimer Paired Helical Filaments: Abnormal Phosphorylation of All Six Brain Isoforns,” Neuron, 8:159-168 (1992a); Ksiezak-Reding, H. et al., “Phosphate Analysis and Dephosphorylation of Modified Tau Associated With Paired Helical Filaments,” Brain Res., 597:209-219 (1992)). In affected neurons, the PHFs are not limited to the cell body; they also may invade the axon and the dendrites, following a breakdown of the normal (mature) segregation of tau to the axon (e.g. Kowall, N. et al., “Axonal Disruption and Aberrant Localization of Tau Protein Characterize the Neuropil Pathology of Alzheimer's Disease,” Ann. Neurol., 22, 639-643 (1987)). In fact, neurites containing PHF have been demonstrated to be continuous with cell bodies that are free of NFT (Schmidt, M. L. et al., “Continuity of Neuropil Threads With Tangle-Bearing and Tangle-Free Neurons in Alzheimer Disease Cortex. A Confocal Laser Scanning Microscopy Study,” Mol. Chem. Neuropathol., 18:299-312 1993)). In the AD brain NFT appear earliest in the entorhinal/transentorhinal cortex and hippocampus to affect memory.

The intracellular progression of the NFT can be monitored by light microscopic examination. Such an examination of neurons in an AD brain reveals neurons filled by NFT to varying degrees. Neurons may present no evidence of NFT, only a whisp of NFT, cell body filled with NFT to varying degrees, or seemingly completely filled with NFT. In addition, neurons may be diffusely immunoreactive to phospho-tau antibodies in the absence of frank NFT. Sumpter et al. (Sumpter, P. Q. et al., “A Quantitative Study of the Ultrastructure of Pyramidal Neurons of the Cerebral Cortex in Alzheimer's Disease in Relationship to the Degree of Dementia,” Neuropath. App. Neurobio., 12(3):321-9 (1986); Sumpter, P. Q. et al., “An Ultrastructural Analysis of the Effects of Accumulation of Neurofibrillary Tangle in Pyramidal Neurons of the Cerebral Cortex in Alzheimer's Disease,” Neuropath. App. Neurobio., 12:305-19 (1986)) report that in biopsy samples of middle temporal gyrus, electron microscopy showed that only 12% of neurons were more than 40% occupied by NFT. In most brain regions, some neurons containing NFT finally die leaving behind only the highly insoluble PHF, now known as a “tombstone” or “ghost” tangle, which are, in fact, the extracellular proteolyzed remnant of tau (Bondareff et al., “Molecular Analysis of Neurofibrillary Degeneration in Alzheimer's Disease. An Immunohistochemical Study,” Amer. J. Path., 137(3):711-23 (1990)). The percent of neurons seen in these various stages depends on the brain region examined and the stage of the disease.

The functional significance of AD-like phosphorylation of tau has been assessed in many studies by determining the effect of phosphorylation on the ability of tau to bind to tubulin (e.g. Alonso, A. C. et al., “Role of Abnormally Phosphorylated Tau in the Breakdown of Microtubules in Alzheimer Disease,” Proc. Nat'l Acad. Sci. U.S.A., 91(12:5562-6 (1994); Lu, Q. et al., “Functional Studies of Alzheimer's Disease Tau Protein,” J. Neurosci., 13:508-515 (1993)), a process that has been thought to be central to the deleterious effects of the abnormal phosphorylation of tau and the consequent disruption-of the cytoskeleton referred to below. The Mandelkows (Gustke. N. et al., “Domains of Tau Protein and Interactions With Microtubules,” Biochemistry', 33(32):9511-22 (1994); Biemat, J. et al., “Phosphorylation of Ser 262 Strongly Reduces Binding of Tau to Microtubules: Distinction Between PHF-Like Immunoreactivity and Microtubule Binding,” Neuron, 11:153-63 (1993)) were able to show by site-directed mutagenesis that the critical phosphorylation site was a previously ignored IGS (serine 262) motif contained within the microtubule-binding domain of tau. These data are currently controversial. The kinase(s) and/or phosphatase(s) responsible for phosphorylation at this site have not yet been definitively identified and sequenced. (See Lovestone, S. et al., “A Critical Stage in Neurodevelopment and Neurodegenerative Processes,” Neurosci., 78:309-324 (1997) for a recent review of tau phosphorylation, the kinases and effects on tau function).

Although the observation that PHF τ is hyperphosphorylated has focused most attention on the kinases, reduced phosphatase activities may play an important role in the altered phosphorylation state of PHF τ (e.g. Goto, S. et al., “Dephosphorylation of Microtubule-Associated Protein 2, T Factor, and Tubulin by Calcineurin,” J. Neurochem., 45:276-283 (1985); Yamamoto, H. et al., “Dephosphorylation of Microtubule Proteins by Brain Protein Phosphatases 1 and 2A, and its Effect on Microtubule Assembly.” J. Neurochem., 50:1614-1623 (1988); Goedert, M. et al., “p42 Map Kinase Phosphorylation Sites in Microtubule-Associated Protein Tau are Dephosphorylated by Protein Phosphatase 2A₁,” FEBS., 312:95-99 (1992b); Harris, K. A. et al., “Okadaic Acid Induces Hyperphosphorylated Forms of Tau Protein in Human Brain Slices.” Ann. Neurol., 33:77-87 (1993)). Additional evidence in support of a role for phosphatase in the abnormal phosphorylation of tau and the formation of NFT comes from a report of reduced low molecular weight acid phosphatase activity recovered from AD brain (Shimohama, S. et al., “Reduction of Low-Molecular-Weight Acid-Phosphatase Activity in Alzheimer Brains,” Ann. Neurol., 33:616-621 (1993)).

Despite the work that has been done, there is little data regarding the nature, the molecular status and the consequences of neurons in the AD brain that are NFT-free or that exhibit other stigmata of the disease (e.g. cathepsin D or tau phospho-epitope immunoreactivity).

It is still not established whether these classical pathologies have a primary causal relationship to the cognitive declines of AD. A number of studies have indicated significant positive relationships between NFT density and cognitive decline (Wilcock, G. K. et al., “Plaques, Tangles and Dementia,” J. Neurol. Sci., 56:343-356 (1982); McKee, A. C. et al., “Neuritic Pathology and Dementia in Alzheimer's Disease.” Ann. Neurol., 30:156-165 (1991); Mena, R. et al., “A Progressive Deposition of Paired Helical Filaments (PHF) in the Brain Characterizes the Evolution of Dementia in Alzheimer's Disease. An Immunocytochemical Study With a Monoclonal Antibody Against the PHF Core,” J. Neuropathol. Exp. Neurol., 50:474-490 (1991); Samuel, W. A. et al., “Severity of Dementia in Alzheimer Disease and Neurofibrillary Tangles in Multiple Brain Regions,” Alz. Disease Assoc. Disorders, 5:1-11 (1991); Bierer, L. M. et al., “Neocortical Neurofibrillary Tangles Correlate With Dementia Severity in Alzheimer's Disease.” Arch. Neurol., 52(1):81-8 (1995); see reviews by Chui, H. C. “A Review Emphasizing Clincopathologic Correlation and Brain-Behavior Relationships,” Dementia, 46:806-814 (1989); Selkoe, D. J., “Biochemistry of Altered Brain Proteins in Alzheimer's Disease,” Ann. Rev. Neurosci., 12:463-490 (1989)). The number of NFT correlates not only with the severity but also with the duration of AD (Arriagada, P. et al., “Neurofibrillary Tangles but Not Senile Plaques Parallel Duration and Severiry of Alzheimer's Disease,” Neurology, 42:631-639 (1992)). Even more recently, data have appeared that indicate that area of entorhinal cortex occupied by SP is equally as good a correlate of cognition in AD as are NFT or other measures studied (Cummings, B. J. et al., “Neurobiology of Aging. Cytoskeletal Neurofilament Gene Expression in Brain Tissue From Alzheimer's Disease Patients. I. Decrease in NF-L and NF-M Message,” J. Ger. Psyc. Neurol., 7(3):153-8 (1996)). It appears probable that the disparities among studies are probably a consequence of fixation and staining methods, how plaques and NFT are defined and quantified as well as characteristics of the patient sample and brain region(s) studied.

However, these data are correlational only, and the mechanisms by which SP or NFT may relate to cognitive decline remains uncertain. Whether it is these lesions, or events associated with these lesions, that provide a mechanism for the brain failure of AD is not known. With regard to NFT in particular, the knowledge of phosphorylation and conformational changes of tau in the absence of NFT (e.g., see IV below) raise the possibility that these may be the changes responsible for the cytoskeletal disruption that destroys the “railroad tracks” for the transport of molecules and organelles within the neuron.

In recent years, there have been developed a number of reagents that appear to indicate stages of disease progression in the cell body. Generally, immunoreactivity to these antibodies appears to represent earlier stages of disease than is indicated by classical methods of revealing NFT. The widespread appearance of neuronal immunoreactivity to cathepsin antibodies (e.g. Cataldo, A. M. et al., “Gene Expression and Cellular Content of Cathepsin D in Alzheimer's Disease Brain: Evidence For Early Up-Regulation of the Endosomal-Lysosomal System,” Neuron, 14(3):671-80 (1995)) in the AD brain suggests an early mobilization of the endosomal-lysosomal system prior to the appearance of NFT. A wide variety of antibodies to specific phosphorylation sites and conformations of tau have been combined in double ICC (with one reaction for NFT) to also indicate that the tau molecule is altered prior to the appearance of NFT. Of particular interest here are several antibodies, including PHF-1 (epitope is phospho-tau at Ser 396/404), and the TG/MC series of antibodies raised in the laboratory of Peter Davies. MC-1 is a conformation dependent tau antibody whose binding requires amino acids 7, 8 and 9 as well as amino acids 312-322 of the tau molecule (Jicha, G. et al., “Alz-50 and MC-1, a New Monoclonal Antibody Raised to Paired Helical Filaments, Recognize Conformational Epitopes on Recombinant Tau,” J. Neurosci. Res., 48:128-132 (1997)). This separation by more than 300 amino acids strongly indicates a conformational requirement. Additionally, phosphorylation, glycosylation, glycation or proteolysis of tau do not appear to be required for MC-1 immunoreactivity. TG-3 has an epitope at phospho Thr 231, with the additional requirement for a β turn. Immuno-electron microscopy has demonstrated that immunoreactivity for both these antibodies proceeds the formation of paired helical filaments. Although TG-3 has been shown to cross-react with MAP-2 under selected circumstances, no cross-reactivity has been. found with MC-1. These antibodies clearly define stages of the development of AD pathology that proceed the formation of NFT.

In addition to the classical neurofibrillary tangles of Alzheimer's disease, there are a host of additional intracellular changes of neurons in AD. These include increased cell death in selected neuronal populations, cytoskeletal disruption, selected deficits in transmitter systems, and loss of synapses.

Selective death of neurons in AD is an established fact. Both neuron counting studies using unbiased stereological methods (e.g. West, M. J. et al., “Differences in the Pattern of Hippocampal Neuronal Loss in Normal Aging and Alzheimer's Disease,” Lancet, 344:769-772 (1994)), as well as older studies using a variety of other methods (see Coleman, P. et al., “Neuron Numbers and Dendritic Extent in Normal Aging and Alzheimer's Disease,” Neurobiol. Aging, 8:521-545 (1987), for review), agree in concluding that there is loss of neurons in AD in excess of any age-related neuronal loss.

In parallel with the more recent literature on loss of neurons in AD, there has developed considerable interest in the mechanism of cell death in AD, with particular attention to apoptosis. Although the time course of apoptosis in vitro suggests that finding an apoptotic neuron in an AD brain that has been diseased for decades is akin to “looking for a needle in a haystack”, there is considerable evidence that apoptotic markers are often found in the AD brain (e.g. Anderson, A. J. et al., “DNA Damage and Apoptosis in Alzheimer's Disease: Colocalization With c-Jun Immunoreactivity, Relationship to Brain Area, and Effect of Postmortem Delay,” J. Neurosci., 16:1710-9 (1996); Smale, G. et al., “Evidence for Apoptotic Cell Death in Alzheimer's Disease,” Exp. Neuro., 133:255-30 (1995); Lassmann, H. et al., “Cell Death in Alzheimer's Disease Evaluated by DNA Fragmentation in situ,” Acta Neuropath., 89(1 ):35-41 (1995); Smale, G. et al., “Evidence for Apoptotic Cell Death in Alzheimer's Disease,” Exp. Neurol., 133:255-30 (1995); Dragunow, M. et al., “In situ Evidence for DNA Fragmentation in Huntington's Disease Striatum and Alzheimer's Disease Temporal Lobes,” Neuroreport, 6:1053-7 (1995); Su, J. H. et al., “Immunohistochemical Evidence for Apoptosis in Alzheimer's Disease.” Neuroreport, 5:2529-33 (1994); but see Behl, C. et al., ” Amyloid Beta Peptide Induces Necrosis Rather Than Apoptosis,” Brain Res., 645:253-64 (1994)). Messages related to apoptosis and/or the cell cycle, such as Bcl-2 (e.g. Vyas, S. et al., “Expression of Bcl-2 in Adult Human Brain Regions with Special Reference to Neurodegenerative Disorders.” J. Neurochem., 69:223-31 (1997); Su, J. H. et al., “Up-Regulation of Bcl-2 is Associated with Neuronal DNA Damage in Alzheimer's Disease,” Neuroreport, 7:437-40 (1996); Su, J. H. et al., “Bax Protein Expression is Increased in Alzheimer's Brain: Correlations with DNA Damage, Bcl-2 Expression, and Brain Pathology,” J. Neurolpath. Exp. Neurol., 56:86-93 (1997)), Bax (e.g. MacGibbon, G. A. et al., “Bax Expression in Mammalian Neurons Undergoing Apoptosis, and in Alzheimer's Disease Hippocampus,” Brain Res., 750:223-34 (1997)), p53 (e.g. Kitamura, Y. et al., “Changes of p53 in the Brains of Patients with Alzheimer's Disease,” Biochem. Biophys. Res. Comm., 232:418-21 (1997)) and p21 ras (e.g. Gartner, U. et al, “Induction of p21 ras in Alzheimer Pathology,” Neuroreport, 6:1441-4 (1995)) have all been shown to be altered in AD. Data such as these raise the question of whether the time course and pattern of gene expression of “apoptosis” (if the potential differences allow the use of that term) in vivo in the AD brain follows the same pattern as that demonstrated in vitro upon NGF withdrawal (Martin. D. P. et al., “Biochemical Characterization of Programmed Cell Death in NGF-Deprived Sympathetic Neurons.” J. Neurobiol., 23:1205-20 (1992); Freeman, R. S. et al., “Analysis of Cell Cycle-Related Gene Expression in Postmitotic Neurons: Selective Induction of Cyclin D1 During Programmed Cell Death,” Neuron, 12(2):343-55 (1994); Johnson, E. M. Jr. et al., “Neuronal Apoptosis: Current Understanding of Molecular Mechanisms and Potential Role in Ischemic Brain Injury,” Journal of Neurotrauma, 12:843-52 (1995)) or other manipulations (Miller. T. M. et al., “Metabolic and Genetic Analyses of Apoptosis in Potassium/Serurn-Deprived Rat Cerebellar Granule Cells,” J. Neurosci., 16:7487-95 (1996)). Additionally, molecules known to exist in the AD brain have been demonstrated to stimulate apoptosis in vitro, including Aβ (Blanc, E. M. et al., “Amyloid-Beta Peptide Induces Cell Monolayer Albumin Permeability, Impairs Glucose TranLsport, and Induces Apoptosis in Vascular Endothelial Cells,” J. Neurochem, 68(5):1870-81 (1997)) and the presenilins (Deng, G. et al., “Gene Expression of Alzheimer-Associated Presenilin-2 in the Frontal Cortex of Alzheimer and Aged Control Brain,” FEBS Letters, 394(1):17-20 (1996)). These in vitro suggestions of the possibility of apoptosis in AD were extended to a demonstration of apoptosis in Aβ expressing transgenic mice (LaFerla F. M. et al., “The Alzheimer's A Beta Peptide Induces Neurodegeneration and Apoptotic Cell Death in Transgenic Mice,” Nature Genetics, 9(1):21-30 (1995)). The in vitro work of Gschwind and Huber (1995) demonstrated that the ability of Aβ to induce apoptosis was dependent on cell type (Gshwind et al., “Apoptotic Cell Death Induced by Beta-Amyloid 1-42 Peptide is Cell Type Dependent,” J. Neurochem., 65(1):292-300 (1995)). A potential mechanism for the relationship between Aβ and apoptosis has been described in in vitro human neurons in which Aβ downregulates Bcl-2 and upregulates bax (Paradis. E. et al., “A. Amyloid Beta Peptide of Alzheimer's Disease Downregulates Bcl-2 and Upregulates Bax Expression in Human Neurons,” J. Neurosci., 16(23):7533-9 (1996)).

As the molecular signaling events of apoptosis gained wider appreciation, the study of mechanisms of apoptosis in AD extended to include the investigation of other molecules associated with apoptosis. The Cotman laboratory described both increased expression of the omnipresent c-Jun in Aβ induced apoptosis in vitro as well as co-localization of c-Jun expression and TUNEL positivity in AD brain (Anderson, A. J. et al., “DNA Damage and Apoptosis in Alzheimer's Disease: Colocalization with C-Jun Immunoreactivity, Relationship to Brain Area, and Effect of Postmortem Delay,” J. Neurosci., 16:1710-9 (1996)). LaFerla (1996) described activation of p53 in response to expression of Aβ by transgenic mice (LaFerla et al., “Extracellular Deposition of Beta-Amyloid Upon p53-Dependent Neuronal Cell Death in Alzheimer's Disease Evaluated bv DNA Fragmentation in situ,” Acta Neuropath., 89(1):35-41 (1995)).

The evidence for a significant relationship between the cell cycle and apoptosis is extensive in the case of dividing cells. The general conclusions derived from this body of work are that dividing cells and apoptotic cells share a number of features. Common morphological features include chromatin condensation, cell rounding and cytoskeletal rearrangements (Ucker, D. S. et al., “Apoptotic Morphology Relects Mitotic-Like Aspects of Physiological Cell Death and is Independent of Genome Digestion,” Micro. Res. Tech., 34(3):267-71 (1996)). In addition, expression of many genes is common to regulation of both the cell cycle and apoptosis. These common genes include (but are not limited to) p53, c-myc. Rb. bcl-2, cyclins—especially cyclin D1 and cyclin dependent kinases (Freeman, R. S., “Cell Death in Diseases of the Nervous System.” Koliatis, eds., Humana Press; Smith, M. L. et al., “Mammalian DNA Damage-Inducible Genes Associated With Growth Arrest and Apoptosis.” Mutation Research, 340(2-3):109-24 (1996); Enoch, T. et al., “Cellular Responses to DNA Damage: Cell Cycle Checkpoints, Apoptosis and the Roles of p53 and ATM,” Trends in Biochem. Sci., 20(10:426-30 (1995); Evan, G. I. et al., “Apoptosis and the Cell Cycle,” Curr. Opin. Cell Biol., 7(6):825-34 (1995); King, K. L. et al., “Cell Cycle and Apoptosis: Common Pathways to Life and Death,” J. Cell. Biochem., 58(2):175-80 (1995); Meikrantz, W. et al., “Apoptosis and the Cell Cycle,” J. Cell. Biochem., 58(2):160-74 (1995) for recent reviews).

However, data are sparse that may be used to support the extension of the concepts derived from dividing cells to neurons, and much of these data come from studies of cell lines or primary cultures of relatively immature neurons (Freeman. R. S. et al., “Analysis of Cell Cycle-Related Gene Expression in Postmitotic Neurons: Selective Induction of Cyclin D1 During Programmed Cell Death,” Neuron, 12(2):343-55 (1994)). Studies based on neurons of human brain that relate apoptosis to cell cycle events are rare, but becoming less so. These data are, essentially, the data demonstrating the expression of cell cycle/apoptosis genes in post mortem human brain in either AD (Nagy, Z. et al., “Cell Cycle Markers in the Hippocampus in Alzheimer's Disease,” Acta Neurorpathologica, 94(1):6-15 (1997); Nagy, Z. et al., “Expression of Cell Division Markers in the Hippocampus in Alzheimer's Disease and Other Neurodegenerative Conditions,” Acta Neuropathologica. 93(3):294-300 (1997); McShea, A. et al., “Abnormal Expression of the Cell Cycle Regulators P16 and CDK4 in Alzheimer's Disease,” Am. J. Path., 150(6):1933-9 (1997); Arendt, T. et al., “Expression of the Cyclin-Dependent Kinase Inhibitor p16 in Alzheimer's Disease.” Neuroreport. 7(18):3047-9 (1996)) or in other diseases (Crino, P. B. et al., “Embryonic Neuronal Markers in Tuberous Sclerosis: Single-Cell Molecular Pathology,” Proc. Nat'l Acad. Sci. U.S.A., 93(24):14152-7 (1996)). Recently, Vincent et al. have extended their earlier observations suggesting mitotic mechanisms in AD by demonstrating expression of Cdc2 and cyclin B1 in tangle-bearing neurons of AD brain, not in neurons of control brain (Vincent et al., “Aberrent Expression of Mitotic Cdc2/Cyclin B1 kinase in Degenerating Neurons of Alzheimer's Disease Brain,” J. Neurosci., 17:3588-98 (1997); Vincent et al., “Mitotic Mechanisms in Alzheimer's Disease?,” J. Cell Bio., 132:413-425 (1996)). The immunohistochemical data of Vincent leave open the vital issues of defining 1) the stage at which these mitotic genes are expressed and 2) the sequence of gene expressions preceeding and following the expression of Cdc2 and cyclin B1.

The disruption of cytoskeleton in AD has been well documented since the 1986 electron microscopic studies of Sumpter (Sumpter. P. Q. et al., “A Quantitative Study of the Ultrastructure of Pyramidal Neurons of the Cerebral Cortex in Alzheimer's Disease in Relationship to the Degree of Dementia,” Neuropath. App. Neurobiol., 12(3):321-9 (1986); Sumpter, P. Q. et al., “An Ultrastructural Analysis of the Effects of Accumulation of Neurofibrillary Tangle in Pyramidal Neurons of the Cerebral Cortex in Alzheimer's Disease,” Neuropath. App. Neurobiol., 12:305-19 (1986)). This conclusion has been supported in more recent studies (e.g. Praprotnik, D. et al., “Filament Heterogeneity within the Dystrophic Neurites of Senile Plaques Suggests Blockage of Fast Axonal Transport in Alzheimer's Disease.” Acta Neuropathologica, 91:226-35 (1996); Braak, E. et al., “A Sequence of Cytoskeleton Changes Related to the Formation of Neurofibrillary Tangles and Neuropil Threads,” Acta Neuroathologica, 87:554-67 (1994); Perry, G. et al., “Neuropil Threads of Alzheimer's Disease Show a Marked Alteration of the Normal Cytoskeleton.” J. Neurosci., 11:1748-55 (1991); see Lee, V. M. “Disruption of the Cytoskeleton in Alzheimer's Disease,” Curr. Opin. Neurobiol., 5:663-8 (1995) for review of earlier studies). In addition, expression of a number of the protein constituents of the cytoskeleton, and their messages has been shown to be altered significantly in AD (e.g. Kittur, S. et al., “Cytoskeletal Neurofilament Gene Expression in Brain Tissue From Alzheimer's Disease Patients. I. Decrease in NF-L and NF-M message,” 'Journal of Geriatric Psychiatry & Neurology, 7:153-8 (1994); Vickers, J. C. et al., “Alterations in Neurofilament Protein Immunoreactivity in Human Hippocampal Neurons Related to Normal Aging and Alzheimer's Disease.” Neurosci., 62:1-13 (1994); Barton, A. J. et al., “Increased Tau Messenger RNA in Alzheimer's Disease Hippocamnpus,” Amer. J. Path., 137:497-502 (1990); see Goldman, J. E. et al., “Cytoskeletal Protein Abnormalities in Neurodegenerative Diseases,” Ann. Neurol., 19:209-23 (1986), for review of earlier studies).

The mechanism for the cytoskeletal disruption in NFT neurons is partially understood. The expression of alpha-tubulin, NF-L and NF-M are all altered in AD (e.g. Brion, J. P. et al., “Distribution and Expression of the Alpha-Tubulin mRNA in the Hippocampus and the Temporal Cortex in Alzheimer's Disease.” Path. Res. Prac., 191(6):490-8 (1995); Kittur, S. et al., “Cytoskeletal Neurofilament Gene Expression in Brain Tissue From Alzheimer's Disease Patients. I. Decrease in NF-L and NF-M Message,” J. Ger. Psy. Neurol., 7:153-8 (1994)). The abnormal phosphorylation of tau in AD brain renders tau extracted from AD brain to be less competent to promote microtubule assembly (e.g. Lu. Q. et al., “Functional Studies of Alzheimer's Disease Tau Protein,” J. Neurosci., 13:508-515 (1993); Alonso, A. C. et al., “Role of Abnormally phosphorylated Tau in the Breakdown of Microtubules in Alzheimer Disease,” Proc. Nat'l Acad. Sci. U.S.A., 91(12):5562-6 (1994)). This inability of tau to promote microtubule assembly leads to failure of normal formation of microtubules (e.g. Gray, E. G. et al., “Alzheimer's Disease Paired Helical Filaments and Cytomembranes,” Neuropathol. Appl. Neurobiol., 13:91-110 (1987); Kowall, N. et al., “Azonal Disruption and Aberrant Localization of Tau Protein Characterize the Neuropil Pathology of Alzheimer's Disease,” Ann. Neurol., 22:639-643 (1987)), thereby disrupting one of the major cytoskeletal highways for some components of anterograde and retrograde transport (e.g. Alberts, B. et al, “Molecular Biology of the Cell,” Second edition. Garland Publishing, New York (1989)).

Expression of messages related to transmitter systems also has been shown to be affected in Alzheimer's disease, including both the cholinergic system (e.g. Boissiere, F. et al., “Decreased TrkA gene Expression in Cholinergic Neurons of the Striatum and Basal Forebrain of Patients with Alzheimer's Disease,” Experimental Neurology, 145(1):245-52 (1997); Mufson, E. J. et al., “Decreased trkA gene Expression within Basal Forebrain Neurons in Alzheimer's Disease,” Neuroreport, 8(1):25-9 (1996)) and excitatory transmitter systems (e.g. Le Prince, G. et al., “Glutamine Synthetase (GS) Expression is Reduced in Senile Dementia of the Alzheimer Type,” Neurochem. Res., 20(7):859-62 (1995); Pellegrini-Giampietro, D. E. et al., “AMPA/Kainate Receptor Gene Expression in Normal and Alzheimer's Disease Hippocampus,” Neurosci., 61(1):41-9 (1994)). NFT have also been demonstrated in a wide variety of subcortical regions, including selected sources of chemically defined diffuse projections to the forebrain including the cholinergic basal forebrain (e.g. Rasool, C. G. et al., “Neurofibrillary Degeneration of Cholinergic and Non-Cholinergic Neurons of the Basal Forebrain in Alzheimer's Disease,” Ann. Neurol., 20(4):482-8 (1986)), the serotonergic nucleus raphe dorsalis (e.g. Yamamoto, T. et al., “Nucleus Raphe Dorsalis in Alzheimer's Disease: Neurofibrillary Tangles and Loss of Large Neurons,” Ann. Neurol., 17(6):573-7 (1985)) and the noradrenergic locus coeruleus (e.g. German, D. C. et al., “Alzheimer's Disease: Neurofibrillary Tangles in Nuclei That Project to the Cerebral Cortex,” Neuroscience, 21(2):305-12 (1987)). NFT also have been shown to accumulate in somatostatin intrinsic cortical neurons (e.g. Davies, P. et al., “Reduced Somatostatin-Like Immunoreactivity in Cerbral Cortex From Cases of Alzheimer's Disease and Alzheimer Senile Dementia,” Nature, 288(5788):279-80 (1980); Kowall, N. W. et al., “Cortical Somatostatin, Neuropeptide Y, and NADPH Diaphorse Neurons: Normal Anatomy and Alterations in Alzheimer's Disease,” Ann. Neurol., 23(2):105-14 (1988)). The transmitter deficits of AD may well be a consequence of the formation of NFT in these chemically defined cell populations, as has been demonstrated for the adrenergic and serotonergic systems in AD (Burke, J. et al., “Evidence for Decreased Transport of PNMT Protein in Advanced Alzheimer's Disease,” J. Am. Geriatr. Soc., 38:1275-1282 (1990); Burke, J. et al., “Evidence for Decreased Transport of Tryptophan Hydroxylase in Advanced Alzheimer's Disease,” Brain Res., 537:83-87 (1990)). NFT in chemically defined cell populations may influence not only transmitters, but additionally, the possible failure of retrograde transport resulting from NFT may lead to the inability of axons to transport trophic factors back to the cell body. This may be of particular importance with regard to the failed maintenance of cholinergic neurons in the basal forebrain in AD.

Synapse loss is, currently, the best correlate of cognitive decline. A relationship of loss of synapses in AD to cognitive decline certainly corresponds to the current understanding of how the brain transmits, processes and stores information through synaptic connections and their modification. Loss of synapses in AD is probably due to 1) inability of a still viable neuron to maintain its full complement of synapses and 2) neuron death. Alternative #1 is likely the more consequential of the two since synapses correlate significantly with cognitive decline while neuron numbers does not (e.g. Terry, R. D. et al., “Physical Basis of Cognitive Alternations in Alzheimer's Disease: Synapse Loss is the Major Correlate of Cognitive Impairment,” Ann. Neurol., 30:572-580 (1991)).

Studies of various brain regions using different methods agree in demonstrating significant loss of synapses in AD. Traditional uranyl acetate and lead citrate staining demonstrated at the e.m. level a 35-42% decrease in synapse density in frontal association cortex of AD brain (area 9) (DeKosky, S. et al., “Synapse Loss in Frontal Cortex Biopsies in Alzheimer's Disease: Correlation With Cognitive Severity,” Ann. Neurol., 27:457-464 (1990)). This study utilized both biopsy and postmortem AD material and showed equivalent synapse losses in both AD groups. Using the same methods, a 23-33% loss of synapses was found in temporal cortex, areas 21 and 22 (Scheff, S. W. et al., “Synapse Loss in the Temporal Lobe in Alzheimer's Disease,” Ann. Neurol., 33:190-199 (1993)). These percentages agree well with the losses found in presumptive areas 9 and 21 (Davies, C. et al., “Quantitative Analysis of the Neuronal and Synaptic Content of the Frontal and Temporal Cortex in Patients With Alzheimer's Disease,” J. Neurol. Sci., 78:151-164 (1987)). Hamos, J. E. et al., “Synaptic Loss in Alzheimer's Disease and Other Dementias,” Neurol., 39:355-361 (1989), quantified synaptophysin and synapsin staining intensity in the dentate gyrus inner and outer molecular layer to demonstrate a roughly three-fold loss of staining intensity in the outer molecular layer. At the RNA level, reductions of the order of 50% in message for synaptophysin, synapsin and synaptobrevin have been observed in the AD-neocortex (Lukiw, W. J. et al., “Cytoskeletal and Synaptic RNA Message Levels in Sporadic Alzheimer's Disease (AD) Neocortex,” Internet Conference on Alzheimer's Disease Sponsored by Alzforum, (1997)). Callahan, L. et al., “Neurons Bearing Neurofibrillary Tangles Are Responsible for Selected Synaptic Deficits in Alzheimer's Disease,” Neurobio. Aging, 16:311-314 (1995), previously demonstrated that this loss of synaptophysin message was largely attributable to tangle-bearing neurons.

In those studies of synapse density in AD in which psychometric data were collected before death, the loss of synapses was found to be well correlated with degree of dementia. Terry, R. D. et al., “Physical Basis of Cognitive Alterations in Alzheimer's Disease: Synapse Loss is the Major Correlate of Cognitive Impairment,” Ann. Neurol., 30:572-580 (1991), found a correlation of −0.76 between scores on the Blessed Information-Concentration-Memory Test and densitometry of synaptophysin staining in middle frontal association cortex (area 46). This is comparable to the correlation of 0.77 between area 9 synaptic density and Mini-Mental Status scores of 8 patients (DeKosky, S. et al., “Synapse Loss in Frontal Cortex Biopsies in Alzheimer's Disease: Correlation With Cognitive Severity,” Ann. Neurol., 27:457-464 (1990)).

The extensive cellular changes in the AD brain discussed above offer ample mechanisms for the cognitive declines of AD. However, many issues remain unresolved. How do these cellular changes relate to the classical markers of disease, the SP and the NFT? Which of these cellular changes are related to molecular markers of the functional potential of a neuron? Are synaptic markers lost in diseased but still viable neurons and, if so, at what stage of the pathological cascade? In what sequence do these cellular changes take place? How are these cellular changes related to molecular changes found in single AD neurons?

In addition to the systems discussed at greater length above message alterations have been demonstrated in a number of other systems in AD brain. Message for mitochondrial enzymes has been shown to be decreased in AD (e.g. Simonian, N. A. et al., “Functional Alterations in Alzheimer's Disease: Selective Loss of Mitochondrial-encoded Cytochrome Oxidase mRNA in the Hippocampal Formation.” J. Neuropath. Exp. Neurol., 53(5):508-12 (1994)), and Chandrasekaran, K. et al., “Impairment in Mitochondrial Cytochrome Oxidase Gene Expression in Alzheimer Disease.” Mol. Brain Res., 24(1-4):336-40 (1994), have suggested that this decrease is preferential to tangle-bearing neurons. Increased expression of lysosomal-endosomal enzymes has been demonstrated in neurons of the AD brain by Nixon and his co-workers (e.g. Cataldo. A. M. et al., “Gene Expression and Cellular Content of Cathepsin D in Alzheimer's Disease Brain: Evidence for Early Up-Regulation of the Endosomal-lysosomal system,” Neuron., 14(3):671-80 (1995)), while Harrison. P. J. et al., “Heat Shock Protein (hsp70) mRNA Expression in Human Brain: Effects of Neurodegenerative Disease and Agonal State,” Neuropath. App. Neurobiol., 19(1):10-21 (1993). demonstrated altered expression of message for heat shock protein. As expected, several laboratories have shown altered expression of presenilin in AD (e.g. McMillan. P. J. et al., “Neuronal Expression of STM2 mRNA in Human Brain is Reduced in Alzheimer's Disease,” J. Histochem. Cytochem., 44(11):1215-22 (1996); Deng, G. et al., “Gene Expression of Alzheimer-Associated Presenilin-2 in the Frontal Cortex of Alzheimer and Aged Control Brain,” FEBS Letters, 394(1):17-20 (1996)). Expression of components of the complement system has been shown to be altered (e.g. Fischer, B. et al., “Complement C1q and C3 mRNA Expression in the Frontal Cortex of Alzheimer's Patients,” J. Mol. Med., 73(9):465-71 (1995)), presumably to play a role related to cell death. Several laboratories have shown decreased expression of genes related to neuronal plasticity, particularly GAP-43/B-50 (e.g. Coleman, P. D. et al., “Reduced GAP-43 Message Levels are Associated with Increased Neurofibrillary Tangle Density in Frontal Association Cortex Area 9 in Alzheimer's Disease,” Neurobiol. Aging, 13:631-639 (1992); de la Monte, S. M. et al., “Aberrant GAP-43 Gene Expression in Alzheimer's Disease,” Am. J. Path., 147(4):934-46 (1995)). This limited survey of high points most pertinent to this proposal omits other molecular changes that have been described, but this review serves the purpose of indicating that a wide variety of molecular changes have been described in the AD brain, and raises the question of which changes take place at similar stages of disease, and what may be the consequences of these molecular changes for the functioning of the brain.

It has been shown that the best correlate of cognitive impairment in Alzheimer's disease (AD) is loss of synapses (DeKosky, S. T. et al., Ann. Neurol., 27:457-464 (1990); Terry, R. D. et al., Ann. Neurol., 30:572-580 (1991)). In situ hybridization studies have demonstrated that neurons with NFT have lower message levels for GAP-43 and for synaptophysin relative to NFT-free neurons (Callahan, L. M. et al., Neurobio. Aging, 16:311-314 (1995)). These losses are selective, since cathepsin D message was shown to be increased in NFT-bearing neurons in the same study. These results indicate a link between the development of NFT and the loss of synapses. NFT contains paired helical filaments (PHF) composed of abnormally phosphorylated tau. However, it remains to be established how (or whether) abnormal phosphorylation of tau leads to the formation of NFT. It is unknown how early in the development of the disease the synaptic alterations occur. The abnormal activation of the endosomal-lysosomal system (Cataldo, A. M. et al., Proc. Natl. Acad. Sci. U.S.A., 88:10998-11002 (1991) has been reported to be an early event in AD. The mRNA for cathepsin D was increased in AD brain in pyramidal neurons of which a majority appeared histologically normal (Cataldo, A. M. et al., Neuron. 14:671-680 (1995)). Other changes in mRNA transcripts in AD brain have been detected including calcium/calmodulin-dependent protein kinase II (Murray, K. D. et al., Neurosci., 60:37-48 (1994)), brain-derived neurotrophic factor (Philips, H. S. et al., Neuron, 7:695-702 (1991); Murray, K. D. et al., Neurosci., 60:37-48 (1994)), extracellular signal regulated kinase 2 (Hyman, B. T. et al., Am. J. Pathol., 144:565-572 (1994)), and GTP binding protein, G (Harrison, P. J. et al., Mol. Brain Res., 10:71-81 (1991)). However, the studies cited above have been limited to examining only one or a few messages at a time by in situ hybridization. Northern blot analysis is another heavily used method to quantify the changes of RNA transcripts in AD (Robinson, C. A. et al., Neurobiol. Aging, 15:681-690 (1994); Duguid, J. R. et al., Ann. New York Acad. Sci., 679:178-187 (1993); Oyama, F. et al., J. Neurchem., 60:1658-1664 (1993); Chandrasekaran, K. et al., Mol. Brain Res., 24:336-340 (1994)). However, these studies provide only a gross view of changes without being able to define the cell types that have the changes. An even more serious drawback of this approach is that without age-matched controls, it is very difficult to tell the age-related changes apart from AD-related changes.

All the previous studies suggest the concept of changing profiles of gene expression as the disease progresses in AD brain. Therefore a need exists for a method which can be used to characterize the molecular events associated with the formation of NFT in single neurons, and to decipher the degenerative process in NFT-bearing cells. Moreover, a need exists for methods of determining molecular markers to diagnose and monitor the treatment of Alzheimer's disease.

### SUMMARY OF THE INVENTION

The present invention provides a method of profiling mRNA production during disease stages. A plurality of cells each of which is characterized by a different stage of disease is isolated. For each of the plurality of cells, antisense RNA transcripts are produced from mRNA and then amplified. The levels of antisense RNA transcripts are measured, where the level of antisense RNA transcript is indicative of the level of the mRNA.

The invention also provides a method for monitoring gene expression in a single cell. The cell is isolated from tissue. Antisense RNA transcripts are produced from mRNA of the cell and then amplified. mRNA levels for individual genes within the cell are measured by quantitating the levels of the antisense RNA transcripts.

Yet another embodiment of the invention is a method of diagnosing or monitoring the progression of a disease. Cells isolated from a subject are classified as diseased or healthy. A diseased or healthy single cell is isolated from the subject. Antisense RNA transcripts are produced from mRNA from the isolated cell and then amplified. mRNA levels are measured in order to quantitate the levels of the RNA transcripts.

### BRIEF DESCRIPTION OF THE DRAWINGS

FIG. 1 depicts a schematic presentation of the single cell approach to mRNA profiling.

FIG. 2 is a photomicrograph of the CA1 hippocampal region of an AD case demonstrating neurons containing NFT neurons (arrowheads), and NFT-free neurons (arrows). Neurons containing differing degrees of NFT-burden (from 0%→75%) are adjacent to each other within the same microenvironment. The studies proposed in this application aim to profile message changes within individual neurons in selected categories to provide further information regarding the response of neurons to the presence of AD.

FIG. 3 shows the separation of cell population from five brains by canonical analysis. The first canonical variable was plotted against the second canonical variable for each cell. Cells from AD brains are filled symbols, and cells from control brains are open symbols.

FIG. 4 shows the results of combined immunocytochemistry/in situ hybridization for synaptophysin messages (FIG. 4A), poly A+ message (FIG. 4B), and cathepsin D message (FIG. 4C). Synaptophysin grain density (FIG. 4D) exhibited a 50% decrease in tangle neurons relative to nontangle neurons. The decrease was specific based on a moderate decrease of poly A+ grain density (FIG. 4E), and a 33% increase of cathepsin D grain density (FIG. 4F) in NFT neurons. These data strongly indicate that profiles of message alterations in individual neurons should be a powerful tool toward the understanding of the response of an individual neuron to the presence of Alzheimer's disease.

FIG. 5 shows double immunocytochemistry combined with in situ hybridization. PHF-1 only neurons (large arrow), NFT neurons (mAb 69+, arrowhead), and neighborhood ICC negative neurons (small arrow). Grain density is for synaptophysin message (grains). Message levels are equivalent between PHF-1 only neurons and ICC negative neighbors.

FIG. 6 shows the results of the double immunocytochemistry experiment to identify neurons which were positive for phosphorylation at tau serine 262, an epitope thought to be critical to the ability of tau to bind microtubules. The immunocytochemistry was combined with in situ hybridization for synaptophysin message. A number of phospho-262 positive neurons showed decreased grain density for synaptophysin message relative to ICC negative neighbors (arrowheads).

FIG. 7A shows acridine orange stained spreads visualized with a fluorescent light source. Intact/viable RNA appears orange and DNA green using this procedure and can be readily identified. Three stained cells with good quality of RNA are indicated by arrows. FIG. 7B is an IHC on human post mortem brain spread using anti NF—H to identify neurons. Two intact NF—H positive neurons are indicated by arrows. Scale bars are 10 μm.

FIG. 8 is a northern gel analysis of radioactively labeled secondary aRNA transcripts. Transcripts from 9 cells (lanes 2-10) isolated from control human post mortem brain are shown. 100 pg of poly(A)+RNA from cultured mouse astrocytes was included as a positive control for all the enzymatic reactions (lane 1). Transcript length ranges from several hundreds bp to 4.4 kb in all 9 samples.

FIG. 9 is a dot blot hybridization of aRNA from one cell with selected cDNAs. The aRNA was used at four concentrations, 2.5×, 1.5×, 1.0×, and 0.5×. For each concentration, hybridization was done in duplicate. On each blot, column a, from rows 1 to 8, the cDNAs are HSP70. p53. HII, nestin, actin, STM2, cyclin D1 and CamK II; column b, rows 1 to 5S182. α1-ACT. GAPDH, GFAP and pBS.

### DETAILED DESCRIPTION OF THE INVENTION

The present invention provides methods for profiling mRNA production during disease stages. A plurality of cells each characterized by a different stage of disease progression are isolated. For each of the plurality of cells, antisense RNA transcripts is produced from the mRNA present in the cells. The antisense RNA transcripts are then amplified. The mRNA levels are measured for each of the plurality of cells bv quantitating the levels of antisense RNA transcripts.

In a preferred embodiment of the invention, the cells are isolated by enzymatically treating the cells. In another embodiment, the isolation of the cells is carried out by laser separation of the cells. Efforts have been made to search for more efficient way to isolate single cells and to seek new techniques for monitoring more genes in a single experiment. A new Laser Capture Microdissection (LCM) technique has been developed (Emmert-Buck, M. R. et al., “Laser Capture Microdissection,'Science, 274(5289):998-1001 (1996), which is hereby incorporated by reference), which will alleviate the tedious time-consuming process of manual isolation of individual cells under microscope. This method will also increase the precision of cell isolation and will allow the simultaneous selection of multiple single neurons with common immunohistochemical characteristics. In yet another embodiment, the Genechip technology is preferred which allows monitoring the RNA levels of thousands of genes in a single experiment (Chee, M. et al., “Accessing Genetic Information With High-Density DNA Arrays,” Science, 274(5287):610-4 (1996), which is hereby incorporated by reference). In this new technique, fluorescent-labeled aRNA is used for hybridization to oligonucleotides synthesized on a glass chip, and all the hybridization, washing and data-reading are automated. Alternatively, a cell may be isolated by placing a small-drop of a liquid, such as water, over the cell and floating the cell away from other cells so that it may be removed.

The quantitation of the antisense RNA transcripts is carried out by dot-blot hybridization of cDNA with the antisense RNA transcripts in a preferred embodiment of the invention. Another method for quantitating of the antisense RNA transcripts is sequencing based serial analysis of gene expression or SAGE (Velculescu, V. E. et al., Science, 270:484-487 (1995), which is hereby incorporated by reference). Yet another method which allows for the rapid screening of numerous samples is high-density cDNA microarray on a glass microscope slide (Schena, M. et al., Science, 270:467-470 (1995) which is hereby incorporated by reference).

Although any cells from a subject may be used preferred cells are isolated from brain tissue. The cells may be isolated from a post mortem sample of cells.

Tissue for in situ hybridization/immunocytochemistry studies can be obtained at autopsy. Postmortem delay of tissue should be limited to 16 hours. Although preferred postmortem times are less than 5 hours. The following blocks of tissue can be obtained at autopsy for single cell studies using immunocytochemistry/in situ hybridization or combinations of those techniques: anterior and posterior hippocampus, posterior cingulate, superior temporal gyrus, middle temporal gyrus, inferior temporal gyrus, superior frontal gyrus, motor cortex, anterior cerebellum, locus coeruleus, substantia nigra, and nucleus basalis.

Tissue is fixed in 4% paraformaldehyde for 24 hours, then undergoes three changes of 30% sucrose in 0.067M phosphate buffer cryoprotectant over the course of 15 days. Following the three changes of sucrose, tissue is rapidly frozen in isopentane precooled to −30° C. (no colder). The tissue is then stored in a sealed container within an airtight bag in a −80° C. freezer. Earlier studies for in situ hybridization indicated that tissue stored for more than 2 weeks in fixative showed decreased ability to obtain message. Therefore, the optimal fixation for quantitative studies was immersion fixation in fresh fixative for 24 hours.

Tissue sections can be prepared by the following method. Prior to sectioning. frozen blocks of interest will be removed from the −80° C. freezer and transported on dry ice to a cryostat precooled to −28° C. The blocks are allowed to equilibrate to −28° C. for approximately 30 minutes. Seventeen to eighteen μm cryostat sections will then be cut and mounted on acid cleaned chrom-alum/poly L-lysine double-coated slides (Simmons, D. M. et al., “A Complete Protocol for In situ Hybridization of Messenger RNAs in Brain and Other Tissue With Radiolabeled Single Stranded RNA Probes,” J. Histochem., 12; 169-81 (1989), which is hereby incorporated by reference). Brain regions to be sampled, case selection and related information have been given above and in the section on Human Subjects. To analyze the probes listed in Table 1, tissue measuring approximately 1.9 cm long will be needed for each case.

Tissue may come from hippocampal formation from well-characterized AD and age-matched controls.

Neurons containing tangles can be identified immunocytochemically. Sections from AD and control brain are processed side-by-side on the same slide. Slides are first processed for demonstration of NFTs or phosphorylated tau, the same slides. are then processed for in situ hybridization (except see double ICC/ISH procedure below).

Antibodies MC-1, TG-3, mAb 69 and cathepsin D can be used to compare message levels in different neuronal disease states. Monoclonal antibodies cathepsin D, TG-3 and MC-1 could be used as markers of presumed earlier stages of AD. The epitopes for these antibodies are discussed in “IV. Newer markers of cellular pathology in AD.” Monoclonal antibody 69 can be used as a marker of frank NFT formation. This antibody reacts to a conformational epitope in NFT.

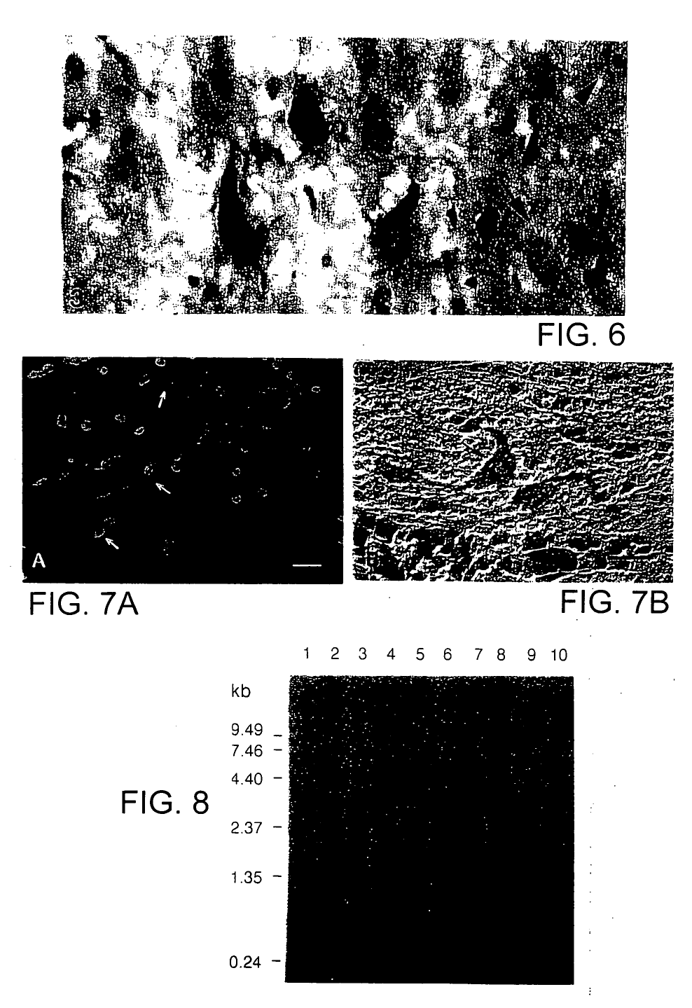

Antibody combinations such as cathepsin D/mAb 69, TG-3/mAb 69, and MC-1/mAb 69 allow analysis of different classes of neurons. Double immunocytochemistry is performed in order to identify populations of neurons which are diff-usely positive for markers of AD but with no frank tangle formation at the light microscopic level. These populations are cathepsin D+/mAb 69−, TG-3+/mAb 69−, and MC-1+/mAb 69−. The double ICC preparations are combined with in situ hybridization with probes for pre and post-synaptic messages, cell stress messages, and cell cycle messages. The method developed involves a DAB reaction of the first antibody, followed by in situ hybridization, then followed by a β-galactosidase reaction for the second antibody. The blue color of the β-galactosidase reaction enables unequivocal identification of neurons that have markers of earlier AD states but that do not have frank tangles at the light microscopic level. Sections which have undergone the DICC/ISH procedure are demonstrated in FIGS. 5A and 6.

Negative and positive controls can also be included for ICC. As negative controls, a set of slides can be included which will not be treated immunocytochemically. Some slides will have no primary antibody. Primary antibody is preabsorbed with purified NFT or other appropriate antigen for other antibodies. In addition, liver tissue on the same slides serve as a negative control for some of the antibodies. Additional negative controls for the double immunocytochemical studies include examination of neurons in serial thin paraffin sections. For positive controls, tangle-staining utilizing mAb 69 in hippocampal/parahippocampal areas from AD tissue are used to confirm ICC procedures are working correctly. Additionally, staining in tissue known to show high levels of protein expression can be used as positive control for other antibodies.

The same slides processed for immunocytochemistry can be processed for in situ hybridization. Preferred probes are described in Table I. A subset of slides will be counterstained with hematoxylin and eosin.

TABLE 1
PROBES FOR IN SITU HYBRIDIZATION STUDIES

| | | Genbank | | | |
|---|---|---|---|---|---|
| | | Account | | | |
| Gene Name | Species | Number | Region | Reference | Reason Chosen |
| Cell Cycle Related Genes - | | | | | |
| (to test hypothesis 1) | | | | | |
| Cyclin D_{1,2,3} | Human | M74092 | Coding | Lew, et al (1991) | Expressed in cell cycle |
| Cyclin D₁ | Human | M64349 | Specific only | Xiong, et al. (1991) | Increased expression in both cell cycle and |
| | | | to Cyclin D₁ | | apoptosis |
| Synaptic Related Genes - | | | | | |
| Presynaptic (to test | | | | | |
| hypothesis 2a) | | | | | |
| Synaptophysin | Human | X06389 | Coding | Cameron, et al (1987) | Synaptic vesicles protein |
| Synaptotagmin p65 | Human | M55047 | Coding | Perin, et al (1991) | Synaptic vesicle sorting protein |
| Synaptobrevin-2 | Human | M36205 | Coding | Archer, et al (1990) | Synaptic vesicle protein |
| | | J05611 | | | |
| Choline Acetyltransferase | Human | S45018 | Coding | May, et al. (1994) | Rate-limiting enzyme of nucleus basalis |
| (CHAT) | | | | | neurotransmitter |
| Tryosine Hydroxylas | Human | Y00414 | Coding | Kaneda, et al (1987) | Rate-limiting enzyme of the locus coeruleus |
| | | X17589 | | | neurotransmitter |
| Synaptic Relate Genes - | | | | | |
| Post-Synaptic (to test | | | | | |
| hypothesis 2b) | | | | | |
| Syntrophin-Beta I | Human | L31529 | Coding | Hugnot, et al (1992) | Post synaptic density protein |
| Neurogranin | Human | X99076 | Coding | Martinez de Arrieta, et al | Dendritic message |
| | | | | (1997) | |
| Cell Stress Related | | | | | |
| Genes (to test hypothesis | | | | | |
| 3) | | | | | |
| Cathepsin B | Human | M14221 | Coding | Chan, et al (1986) | Cell stress, aRNA indicates an increase in |
| | | L16510 | | Cao, et al (1994) | Alzheimer's Disease |
| Cathepsin D | Human | M11233 | Coding | Faust, et al (1985) | Cell-stress, shown to increase in Alzheimer's |
| | | | | | Disease |
| Heat Shock protein 27 | Human | L39370 | Coding | Hickey, et al., (1986) | Cell-stress, indications from aRNA of increase |
| | | X03900 | | Fuqua, et. al., (1989) | in Alzheimer's Disease |
| | | P04792 | | | |

ANTIBODIES

| | | | Epitope | | | |
|---|---|---|---|---|---|---|
| Antibody | Species | Type | Location | Reference | Source | Reason Chosen |
| mab 69 | mouse | monoclonal | PHF, core | Yen, et al., 1987 | Yen | Recognizes phosphorylation-independent epitope in |
| | | | region | | | core of PHF |
| PHF-1 | mouse | monoclonal | Tau, 389-402 | Lane, et al., 1992 | Davies | Recognizes abnormally phosphorylated tau |
| Cathepsin | sheep | polyclonal | N/A | Cataldo, et al., 1990 | Nixon | Recognizes lysosomal enzyme increased in AD |
| TG-3 | mouse | monoclonal | tau phospho thr | Vincent, et al., | Davies | Recognizes tau at phospho-antigen expressed in |
| | | | 231 and B turn | (1996) | | miototic cells |
| | | | requirement | | | |
| MC-1 | mouse | monoclonal | AA 7, 8, 9 and | Jicha, et al (1997) | Davies | Recognizes tau in early abnormal confirmation |
| | | | AA 312-322 of | | | |
| | | | tau | | | |

Riboprobes are preferred for these analyses since 1) it is easily possible to alter the signal to noise ratio of the probe by altering the specific activity of the riboprobe, 2) in the case of low copy messages an adequate signal to noise ratio can be expected, and 3) extensive experience in riboprobe methodologies exist.

cDNAs for the riboprobes to be used are either obtained from others as indicated or derived by RT-PCR based on published sequences, such as those found in GenBank. using mRNA extracted from human brain material. Other probes of interest may be added s they are described as being potentially related to AD and NFT and as sequence is available. Probes can, if necessary, be hydrolyzed to yield a probe size of ˜200 base pairs. Purification can be accomplished with the RNaid Plus System. Size analysis %ill be performed by gel electrophoresis for RNA. Table I summaries the information for several preferred probes for in situ hybridization studies. Preferred messages are those related to: 1) cell cycle, 2) pre- and post-synaptic proteins, and 3) cell stress. The specific proteins listed in the table were chosen based on extensive characterization of the message and/or protein in the literature, and low homology to known proteins.

To ensure that the cDNA fragments chosen will yield riboprobes specific only to the message of interest, a full homology search is performed for each potential message of interest. The names of the selected genes are searched in the NCBI database which is maintained by the National Institute of Health (NIH) (http://www.ncbi.nlm.nih.gov/dbEST/index.html). This database is a compilation of all; known genes, their sequences, their discoverer, their sequencing orientation and other relevant information. Each entry is given a Genbank accession number which is used for identification purposes. This accession number is also used by various computer programs which link to the NCBI database for analysis of the DNA sequences, amino acid sequences, protein translations, and numerous other tasks. Versa Term PRO and Mac Vector, two programs which link to the NCBI database, were used to generate the information in Table 1.

Potential genes are used as keywords for a search in the NCBI database to return all files which contained these names. The accession numbers specific for genes that were in the species homo sapien were used to “fetch” files in Versa Term Pro to retrieve the Genbank file for that gene. The Genbank file includes complete nucleotide sequences, reference information, and other information relevant to ordering and production of the gene.

Homologies of the retrieved nucleotide sequences are performed by a “blast” search in Mac Vector, which searches all known sequences in the Genbank files. The purpose of this search is to return all known genes having homology to the selected gene, rank them in order from highest to lowest homology, and to present a display of the region over which the selected genes and homologous sequence are matched. It is important to note that the sequences to which the gene is searched against are only those which are currently known. The final cDNA fragment chosen to be used for the riboprobe must: 1) show no or very little homology to the known sequences, 2) be capable of being cloned into a transcription vector, such as Bluescript, 3) have a GC content of approximately 50% since higher GC content increases probe stickiness and background, and lower GC content (below 40%) tends to yield probes that are less predictable in the stringency washes), and 4) the fragment needs to be at least 200 bp in length.

cDNAs can be verified by sequencing each end and restriction digest mapping to confirm that the plasmid obtained contains the sequence expected.

Messages proposed for this method are stable during the postmortem intervals proposed. A number of studies have indicated that mRNA is relatively stable postmortem (e.g. Morrison, M. et al., “The Isolation and In Vitro Translation of Undegraded Messenger RNAs From Human Postmortem Brain,” Anal. Biochem., 113:318-24 (1981); Johnson, S. et al., “Extensive Post-Mortem Stability of RNA From Rat and Human Brain,” J. Neuroscience Res., 16:267-80 (1986); Coleman, P. D. et al., “Reduced GAP-43 Message Levels Are Associated With Increased Neurofibrillary Tangle Density in Frontal Association Cortex Area 9 in Alzheimer's Disease,” Neurobiol Agung, 13:631-639 (1992), which are hereby incorporated by reference). On the other hand, one laboratory has presented data indicating that each mRNA species may have unique decay kinetics in postmortem human brain. GFAP and a-tubulin were found to have half lives longer than the longest postmortem interval evaluated (13.5 hours), while the half life for low m.w. neurofilament was 6.5 hours, and for β actin 12 hours (Lukiw, W. et al., “Cytoskeletal Messenger RNA Stability in Human Neo-Cortex: Studies in Normal Aging and Alzheimer's Disease,” Internatl. J. Neurosci., 55:81-8 (1990), which is hereby incorporated by reference). The signal level can be compared in adjacent NFT or otherwise marked AD neurons and NFT-free neurons within the same sections, thus controls for postmortem degradation are built in. AD and control sections should be matched on the same slide to control for postmortem delay. A regression analysis of the data can be carried out against postmortem delay to determine if this variable affected the results.

Controls for in situ hybridization may also be utilized. As negative controls, the sense strand will be obtained as described. In addition, sections of liver will be processed alongside sections of brain to serve as a negative control for neuron specific messages. The standard positive control for human fixed tissue processed for in situ hybridization by the above methods is synaptophysin message in hilus and CA3/CA2 of age matched control hippocampus. Tissues expected to have high message level will be used as positive controls for other probes.

Alternatively, the cells may be isolated from a sample collected from a living patient. Preferred samples include blood, cheek scrapings, cerebral spinal fluid, saliva, urine, and skin.

Amplification of a selected, or target, nucleic acid sequence may be carried out by any suitable means. (See generally Kwoh, D. and Kwoh, T., Am. Biotechnol Lab., 8, 14 (1990) which is hereby incorporated by reference.) Examples of suitable amplification techniques include, but are not limited to, polymerase chain reaction, ligase chain reaction (see Barany, Proc. Nat'l Acad. Sci. U.S.A., 88, 189 (1991), which is hereby incorporated by reference), strand displacement amplification (see generally Walker, G. et al., Nucleic Acids Res, 20, 1691 (1992); Walker,G. et al., Proc. Nat'l Acad. Sci. U.S.A., 89, 392 (1992), which are hereby incorporated by reference), transcription-based amplification (see Kwoh, D. et al., Proc. Nat'l Acad. Sci. U.S.A., 86, 1173 (1989), which is hereby incorporated by reference), self-sustained sequence replication (or “3SR”) (see Guatelli, J. et al., Proc. Nat'l Acad. Sci. U.S.A., 87, 1874 (1990), which is hereby incorporated by reference), the Qb replicase system (see Lizardi, P. et al., Biotechnology, 6, 1197 (1988), which is hereby incorporated by reference), nucleic acid sequence-based amplification (or “NASBA”) (see Lewis, R., Genetic Engineering News, 12(9), 1 (1992), which is hereby incorporated by reference), the repair chain reaction (or “RCR”) (see Lewis, R., Genetic Engineering News, 12(9), 1 (1992), which is hereby incorporated by reference), and boomerang DNA amplification (or “BDA”) (see Lewis, R., Genetic Engineering News. 12(9), 1 (1992), which is hereby incorporated by reference). Polymerase chain reaction is currently preferred.

In general, amplification techniques such as the foregoing involve the use of a probe, a pair of probes, or two pairs of probes which specifically bind to the nucleic acid molecule of interest, but do not bind to other nucleic acid molecules, under the same hybridization conditions, and which serve as the primer or primers for the amplification reaction.

The method described above may also include the steps of measuring mRNA levels by quantitating the levels of antisense RNA transcripts for control genes and then comparing the mRNA levels for the transcripts to the mRNA levels for the control gene transcripts using multivariate analysis. The use of control levels allows for the rapid identification of mRNA transcripts which are aberrantly expressed in the diseased cells.

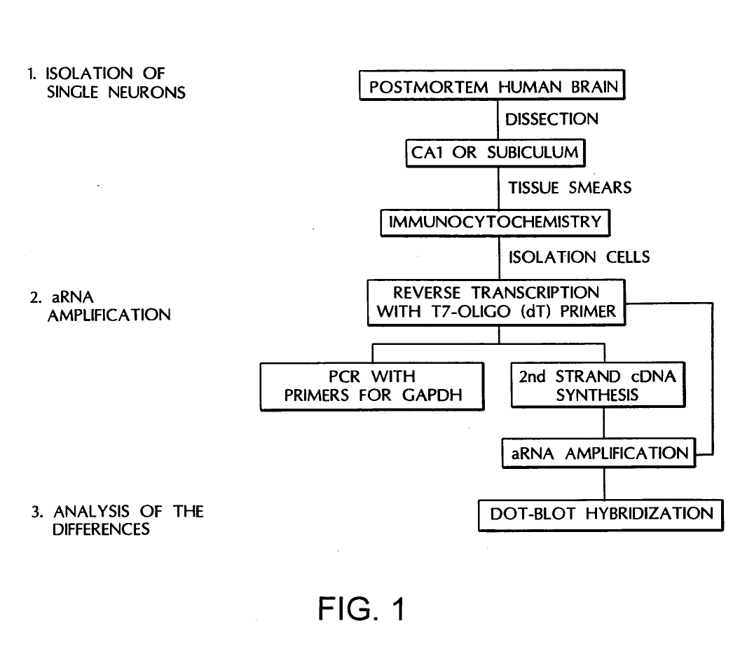

The method is broadly applicable to any disease, including infections by bacteria, fugi, or viruses; genetic diseases; autoimmune diseases; and degenerative diseases. But, in a preferred embodiment, the present invention is used to monitor mRNA transcript levels in Alzheimer's disease. In Alzheimer's disease, the preferred cells to be monitored are neurofibrillary tangle cells. A schematic presentation of the single cell approach as applied to Alzheimer's disease is provided in FIG. 1.

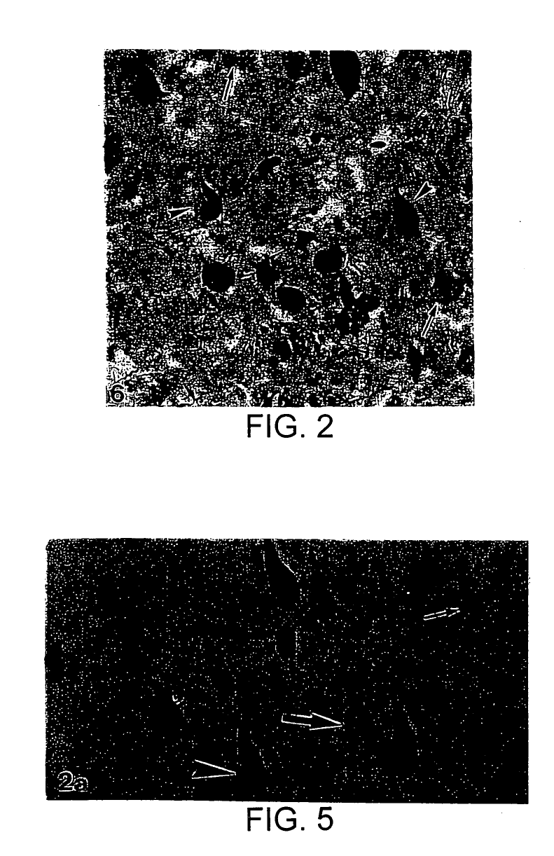

The present invention allows the of the role of neurons in different stages of AD derives from a consideration of the microscopic examination of AD brains which reveals neurons filled with neurofibrillary tangles (NFT) to varying degrees existing side-by-side with neurons apparently free of NFT. FIG. 2 demonstrates this phenomenon in which NFT neurons are stained brown (arrowheads) and NFT-free neurons are stained only with the H&E counterstain (arrows). Neurons which contain NFT and other known markers of AD (presumably at earlier stages) will be identified immunohistochemicallv with antibody to reveal NFT and/or the selected marker antibodies. Combining this detection of disease stage markers with in situ hybridization for selected messages of interest will allow for the comparison of levels of messages of interest in neurons in different stages of the disease with the same message in adjacent unaffected neurons. Additionally, analysis of control brain processed side-by-side with AD brain sections will enable one to compare expression in tangle-free neurons in AD brain with expression in tangle-free neurons in control brain. The invention has significant advantages over the use of Northern blot analysis, since valuable single cell information is lost in analyses which homogenize blocks of tissue.

Preferred RNA transcripts are encoded by a gene selected from the group consisting of (1) synaptic markers, (2) lysosomal hydrolases, (3) kinases and phosphatases, (4) neurotrophic factors, (5) cell cycle regulators, (6) apoptosis factors, (7) mitochondrial genes, and (8) other proteins associated with Alzheimer's disease.

1. Synaptic Markers

As mentioned above the best correlate of cognitive impairment in Alzheimer's disease (AD) is loss of synapses. Studies by in situ hybridization have demonstrated that neurons with neurofibrillary tangles have lower message levels for GAP-43 and for synaptophysin relative to NFT-free neurons (Callahan, L. M. et al., Neurobio. Aging, 16:311-314 (1995), which is hereby incorporated by reference). In addition to these two genes, additional synaptic markers include synapsin I and synapsin II (Süidhof, T. C. et al., Science, 245:1474-1479 (1989), which is hereby incorporated by reference).

2. Lysosomal Hydrolases

According to Cataldo, A. M. et al., Proc. Natl. Acad. Sci. U.S.A., 88:10998-11002 (1991), which is hereby incorporated by reference, the endosomal-lysosomal system is abnormally activated in AD. By using in situ hybridization, they demonstrated that the mRNA for cathepsin D was increased in AD brain in pyramidal neurons of which a majority appeared histologically normal (Cataldo, A. M. et al., Neuron, 14:671-680 (1995), which is hereby incorporated by reference). Other lysosomal hydrolases include cathepsin B, cathepsin L, and β-hexosaminidase A.

3. Kinases and Phosphatases

Hyperphosphorylation of tau could result from aberrations in protein phosphorylation of dephosphorylation, or both. In vitro studies indicate that tau protein can be phosphorylated by a wide range of kinases to produce PHF phospho-epitopes, including ERK/MAP kinases (Drewes, G. et al., EMBO J., 11:2131-2138 (1992); Ledesma, M. D. et al., FEBS Lett., (1992); Goedert, M. et al., FEBS Lett., 312:95-99 (1992); Lu, Q. et al., J. Neurosci., 35:439-444 (1993), which are hereby incorporated by reference), cdc2-like kinases (Mawal-Dewan, M. et al., J. Biol. Chem., 267:19705-19709 (1992); Bauman, K. et al., FEBS Lett., 336:417-424 (1993); Paudel, H. K. et al., J. Biol. Chem., 268:23512-23518 (1993); Ishiguro, K. M. et al., J. Bio. Chem., 267:10897-10901 (1992), which are hereby incorporated by reference), glycogen synthase kinase (Hanger, D. P. et al., Neurosci. Lett., 147:58-62 (1992); Mandelkow, E.M. et al., FEBS Lett., 394:315-327 (1992), which are hereby incorporated by reference), and protein kinase p110^{mark} (Drewes, G. et al., J. Bio. Chem., 270:7679-7688 (1995), which is hereby incorporated by reference). Protein phosphatases PP-2B, PP-2A and PP-1 have been shown to dephosphrylate tau in vivo (Gong, C. et al., FEBS Lett., 341:94-94 (1994); Gong, C. et al., J. Neurochem., 62:803-806 (1994b), which are hereby incorporated by reference).

In AD brain, ERK2 and mRNA has been shown to be decreased in NFT-bearing neurons (Hyman, B. T. et al., Am. J. Pathol., 144:565-572 (1994), which is hereby incorporated by reference), while calcium/calmodulin-dependent protein kinase II was increased (Murray, K. D. et al., Neurosci., 60:3748 (1994), which is hereby incorporated by reference). Other reported changes include decreased casien kinase 2 immunoreactivity (Aksenova, M. V. et al., FEBS Lett., 279:55-57 (1992); Masliah, E. et al., Am. J. Pathol., 140:263-268 (1992), which are hereby incorporated by reference), decreased phosphatidylinositol (PI) kinase and PI phosphate kinase activities (Jolles, J. et al., J. Neurochem., 58:2326-2329 (1992), which is hereby incorporated by reference), reduction of the zinc- and magnesium-stimulated protein tyrosine kinase activity (Vener, A. et al., FEBS Lett., 328:6-8 (1993), which is hereby incorporated by reference), and decrease in activities of phosphoseryl/threonyl protein phosphatases (PPs)-1 and -2A (Gong, C. et al., J. Neurochem., 61:921-927 (1993), which is hereby incorporated by reference).

4. Neurotrophic Factors

Decreased transcript abundance of brain-derived neurotrophic factor (BDNF) has been detected in AD brain by in situ hybridization (Phillips, H. S. et al., Neuron., 7:695-702 (1991); Murray, K. D. et al., Neurosci., 60:37-48 (1994), which are hereby incorporated by reference). However, it was not addressed in these studies if all neurons in AD brain have lower level of BDNF expression compared to neurons in control brain, or only NFT-bearing neurons are responsible for the decrease of BDNF message. In the study by Phillips, H. S. et al., Neuron., 7:695-702 (1991), which is hereby incorporated by reference, the levels of nerve growth factor (NGF) and neurotrophin 3 (NT-3) staining of transforming growth factor-β2 (TGF-β) in NFT-bearing neurons (Flanders, K. C. et al., Neurology, 45:1561-1569 (1995), which is hereby incorporated by reference).

5. Cell Cycle

Accumulating evidence suggests that aberrant regulation of cell cycle occurs in AD brain. A recent study by Vincent, I. et al., J. Cell Bio., 132:412-425 (1996), which is hereby incorporated by reference, showed that the phospho-epitopes recognized by seven new anti-PHF monoclonal antibodies are also detectable in M phase of the cell cycle. In the same study, they showed that a monoclonal antibody MPM-2, specific for mitotic phospho-epitopes (Davis, F. M. et al., Proc. Natl. Acad. Sci. U.S.A., 80:2926-2930 (1983), which is hereby incorporated by reference), stained NFT and neuritic plaques in AD brains, and that no staining was observed with control brains. Moreover. immunoreactivity to S phase markers, p105 (Masliah, E. et al., Lab Invest., 69:562-568 (1993), which is hereby incorporated by reference) and Ki67 (Smith, T. W. et al., Neuropathol. Exp. Neurol., 54:297-303 (1995), which is hereby incorporated by reference), and to cdc2kinase (Wood, J. G. et al., Neurosci. Lett., 156:83-86 (1993);

Liu, W.-K. et al., Am. J. Pathol., 146:228-238 (1995), which are hereby incorporated by reference) have also been reported in AD.

6. Apoptosis

It has been reported that β-amyloid induced apoptosis in cultured cells (Loo. D. T. et al., Proc. Natl. Acad. Sci. U.S.A., 90:7951-7955 (1993), which is hereby incorporated by reference) and in transgenic mice (LaFerla, F. M. et al., Nature Genetics, 9:21-30 (1995), which is hereby incorporated by reference). In a recent study,-a mouse homologue of STM2, the familial AD gene on chromosome 1 (Levy-Lahad. E. et al., Science, 269:973-977 (1995), which is hereby incorporated by reference), was found to confer resistance to T cell receptor-induced cell death (Vito, P. et al., Science, 271:521-525 (1996), which is hereby incorporated by reference). Therefore, it has been speculated that apoptosis might play a role in neurodegeneration in AD.

In recent years, a lot has been learned about the apoptotic pathway. There is evidence suggesting that several regulators of the cell cycle might also be involved in the control of apoptosis (reviewed by Ross, M. E., Trends Neurosci., 19:62-68 (1996), which is hereby incorporated by reference). If the expression of the cell cycle regulators is abnormal in terminally differentiated neurons, instead of leading to abnormal proliferation, it could drive the apoptotic pathway. These include tumor. suppressors p53 and pRb, oncogenes like c-myc and c-jun, cyclin D1 and cdc2.

7. Mitochondrial Genes.

A decrease of mRNA for mitochondri al-encoded cytochrome oxidase (COX) subunits I and III (Chandrasekaran, K. et al., Mol. Brain Res., 24:336-340 (1994), which is hereby incorporated by reference) in AD brain has been reported.

8. Other Proteins Associated with Alzheimer's Disease

Several other proteins are also associated with AD. Tau protein and β-amyloid precursor protein (APP) are implicated in the pathogenesis of AD. Previous study found no significant differences in tau and APP mRNA levels between AD and control brains (Oyama, F. et al. J. Neurochem., 60:1658-1664 (1993). which is hereby incorporated by reference).

α₁-antichymotrypsin (ACT), a component of senile plaques, has been shown to have an increased expression in some cases of AD (Duguid, J. R. et al., Annals New York Acad. Sci., 679:178-187 (1993); Abraham, C. R. et al., Cell, 52:487-501 (1988), which are hereby incorporated by reference). In addition, discordance was found between ACT staining and NFT staining (Duguid, J. R. et al., Annals New York Acad. Sci., 679:178-187 (1993), which is hereby incorporated by reference), which suggests that elevation of ACT expression precedes NFT formation.

Several developmental stage specific markers, i.e. nestin (Lendahl, U. et al., Cell. 60:585-595 (1990); Dahlstrand, J. et al., J. Cell Sci., 103:589-597 (1992), which are hereby incorporated by reference), c-series gangliosides, and microtubule-associated protein 5 (Takahashi, H. et al., Acta Neuropathol., 81:626-631 (1991), which is hereby incorporated by reference) are also preferred.

The present invention also provides a method for monitoring gene expression in a single cell, where the cell is isolated from tissue. The cell is isolated from tissue as described above. Antisense RNA transcripts are produced from the mRNA within the cell. The antisense RNA transcripts are then amplified. mRNA levels are measured for individual genes within the cell by quantitating the levels of the antisense RNA transcripts.

In a preferred embodiment of the invention, the mRNA level for a gene expressed in a cell is compared to the mRNA level for the gene expressed in a second cell.

In another embodiment, mRNA levels may be measured by quantitating the levels of antisense RNA transcripts for control genes. The mRNA levels for the transcripts of interest are compared to the mRNA levels for the control gene transcripts using multivariate analysis. This allows for the selection of transcripts whose level of expression is altered in the cell.

In an one embodiment of the invention the first and second cells are at different stages of development. In another embodiment, one cell is diseased while the. second cell is healthy. In a preferred embodiment, the disease is Alzheimer's disease.

In yet another embodiment, the first cell is exposed to an experimental compound and the second cell is exposed to a different or no compound. In a more preferred embodiment, the compound is an experimental drug. Alternatively, the compound is an environmental toxin. In addition to testing the effects of compounds, the present invention may be used to monitor the effects of an environmental stimulus on the level of mRNA transcripts. One preferred environmental stimulus is radiation.

Another embodiment of the invention is a method of diagnosing or monitoring the progression of a disease. Cells are classified as diseased or healthy. Single cells which are classified as diseased are isolated from a subject. Antisense RNA transcripts are produced from the mRNA within the cell. The antisense RNA transcripts is then amplified. mRNA levels are quantitated by measuring the RNA transcripts.

The single cell approach can be used to identify and quantify aRNA in multiple cells of each brain, two brains of each type (AD vs. control). Values (counts) for each dot on the membrane are manipulated by subtracting the value from a blank spot. Then the values obtained by aRNA probes are normalized to the values obtained by the T7 oligo probe for the corresponding spot. In order to compare across samples, the signals are also normalized to the mean of all the measurements.

As described in the examples, the data were subjected to analysis of variance (ANOVA). If the difference between the observed and predicted mean squares was over 3 for a cell, the cell was defined as an outlier and removed. The results show that the changes at RNA level are (1) significant (p<0.05) for (1-ACT, cyclin D1, HSP27, glutamate decarboxylase (GAD), Wee1, and HES1; (2) marginal (0.1<p<0.05) for GAPDH, nestin, cathepsin β, fos-related antigen 1(Fra-1), crystallin, and ras-L, TC25; and (3) not significant for CamK II, presenillin 1, HSP90, NF-M, tuberin, inhibitor of apoptosis (IAP), ferritin L and H, cyclin B1, cyclin G1, cyclin E, HSP70, Bax, ras-L, TC4, and CDK4. The expression levels for cyclin D1, HSP27, GAD, Wee1 and HES1 are significantly decreased in AD, and that for α1-ACT increased. Among the 6 genes with marginal changes, levels are reduced for GAPDH, nestin, crystallin, and increased for cathepsin B, fra-1, and ras-L, TC25.

The variability can be estimated for each of the factors tested in the experiments, namely, replication (each cDNA spotted twice) on a blot, cell to cell variation within a brain, brain to brain variation and disease. The specific portions of the total variance assigned to the four levels of variation average 62%, 16%, 4.4%. and 17.6%, respectively. In general, replication is the biggest source of variation. Despite the efforts to reduce the experimental error by normalization, there are other factors which may effect the error level. Therefore, statistical analysis is used to differentiate real difference from background noise. The variation for cells within the same brain most of the time is larger than variation for brains within the same group. For genes whose expression levels are significantly different between control and AD, the variation at the disease level then accounts for a larger portion of the total variance.

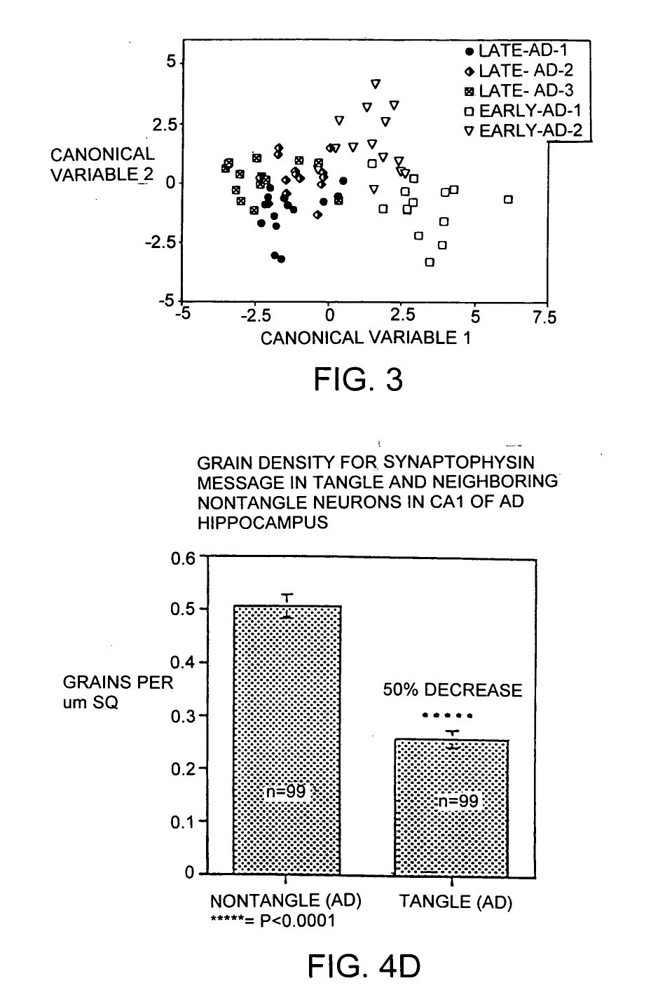

By considering all of the parameters (cDNAs) in a multivariate analysis, it is possible to derive additional information about altered levels of message expression of single cells due to individual brain differences and to disease vs. control differences. Canonical analysis (Kshirsager, A. M. “Multivariate Analysis,” Mariel Dekker, Inc., New York (1972), which is hereby incorporated by reference) has been used to define weighted linear combinations of cDNAs that distinguish brains. Applying this analysis to data from single pyramidal neurons from subiculum of 5 brains (three AD and two controls) yields the result shown in FIG. 3. Each point in this two-dimensional plot represents one cell from a defined brain and each brain is indicated by a different color. Canonical variable 1 represents the weighted combination of cDNAs that best distinguished among the five brains. Canonical variable 2 is an analysis of residual variation among brains, and clearly does not add greatly to the analysis. Brains in blue, red and gray are AD brains, and their cells can be seen to cluster together in the two-dimensional space of this multivariate analysis. Brains in black and green are control brains, and their cells are separated from the cluster formed by AD brains, as well as from each other, in this analysis. This canonical analysis has been repeated in another experiment with data obtained from different brains with similar results. The results of ANOVA analysis and of canonical analysis reinforce each other, with the canonical analysis providing a more graphic presentation. The combination of both types of analyses leads to the conclusion that the single cell aRNA method is capable of revealing differences in message expression due to brains and to disease.

Chi-square analysis can also be used to test statistical differences. Since the data will be counts of grains over classes of cells, a major statistical tool for data evaluation will be the Chi-square test. The model established for the Chi-square test is one which assumes that grain densities over neurons will be equal regardless of whether or not they contain NFT or early disease marker (to any degree) or not, and regardless of whether the neurons are sampled from AD or control brain.

The present invention focuses on the examination of messages in single neurons, as opposed to proteins for a variety of reasons. The most compelling being that many of the proteins of interest are transported a significant distance from the cell body (i.e. to the axon, the synapse and the dendrites). Messages, on the other hand, are localized to the cell body, with the exception of some minor amount of message in the dendrite (e.g. Steward, O. et al., “Protein-Synthetic Machinery at Post-Synaptic Sites During synaptogenesis: A Quantitative Study of the Association Between Polyribosomes and Developing Synapses,” J. Neurosci., 6:412-23 (1986), which is hereby incorporated by reference), and can therefore be related to whether the cell body contains NFT. Complete understanding of the cellular responses of any protein requires knowledge of events at all stages from transcription and its regulation, to translation, to posttranslational processing, translocation and subsequent events.

### EXAMPLES

### Example 1

### Methods

Collection of Tissue: The single cell approach (FIG. 1) was based on the methods of Eberwine, J. et al., “Analysis of Gene Expression in Single Live Neurons,” Proc. Natl. Acad. Sci. U.S.A., 89:3010-3014 (1992), which is hereby incorporated by reference) with modifications in tissue processing. Human brain tissue for this study was obtained from the Rochester Alzheimer's Disease Center. Criteria inclusion as a normal control case have been previously reported (Cheetham, J. E. et al., “Gap-43 Message Levels in Anterior Cerebellum in Alzheimer's Disease,” Mol. Brain Res., 36:145-151 (1996), which is hereby incorporated by reference). Human brain tissue was collected directly at autopsy with a maximum post mortem delay of 16 hours. The samples were transferred into ice cold phosphate-buffered saline (PBS) and maintained on ice until processed for cell spreads.

Preparation of cell spreads and storage: The CA1 or subiculum regions of post mortem human hippocampus were dissected out. Tissue was cut into approximately 1 mm³ blocks and pretreated with 0.25% trypsin (prewarmed to 37° C.) for 20 minutes. The trypsin was aspirated and any remaining was diluted out with ice cold PBS. A single tissue block was transferred to a microscope slide subbed with Cell-Tak (Collaborative Biomedical Products). A siliconized slide was placed on top and with very slight pressure, the top slide was moved over the bottom to spread the tissue. It is important not to use too much pressure, which could result in loss of RNA. These spreads were then fixed with 70% ethanol/150 mM NaCl for 5-10 minutes. Spreads were then either rehydrated to PBS and processed immediately for single cell isolation or dehydrated by rinsing with an ascending ethanol series. The slides were allowed to air dry for 5 minutes, transferred to slide mailers when dry and stored at −80° C. until further use. Al solutions and equipment were treated to render them RNase free.

Acridine orange staining: Acridine orange staining was carried out to indicate whether the previous procedure had degraded the RNA. Cell spreads were stained with acridine orange (Churukian, C. J. “Manual of The Special Stains Laboratory, Dept. of Pathology and Laboratory Medicine, University of Rochester Medical Center, Rochester, N.Y., p159 (1974), which is hereby incorporated by reference) (Molecular Probes). Spreads were covered with 1% acetic acid for 1 minute, rinsed 2×30 seconds with distilled water, stained in 0.02% acridine orange in 0.067M phosphate buffer (pH 6) for 30 seconds, then rinsed 2×30 seconds in 0.067M phosphate buffer (pH 6), differentiated in 0.1M calcium chloride for 45 seconds and rinsed again 2×30 seconds in 0.067M phosphate buffer (pH 6). Finally spreads were mounted under a coverslip with 0.067M phosphate buffer (pH 6) and the orange/green fluorescence visualized with a mercury lamp and a Nikon B2 filter. Cells for RNA profiling were isolated from unstained slides.

IHC: Standard IHC was carried out using anti-Neurofilament 200 kDa (Boehringer Mannheim) antibody to confirm neuronal identity (Shaw, G. et al., “Reactivity of a Panel of Neurofilament Antibodies on Phosphorylated and Dephosphorylated Neurofilaments,” Eur. J. of Cell Biol., 42:1-9 (1986), which is hereby incorporated by reference). Triton-X-100 was not used. To avoid RNase contamination antibodies were pre-incubated for 15 minutes at room temperature with 10% RNase inhibitor (PRIME, 5′-3′), acetylated BSA (200 mg/ml) was substituted for serum in the blocking step, and all equipment used was pretreated to remove RNase. Incubation with the primary antibody was carried out for 1 hour at room temperature (anti NF—H). Primary and secondary antibody incubations were performed under coverslips to conserve volume. Immunoreactivity was detected using a horse anti mouse HRP labeled secondary antibody and the chromagen 3,3′-Diaminobenzidine Tetrahydrochloride (DAB).

Isolation of cells: Individual cells were isolated using an inverted microscope mounted on a vibration isolation table. A finely drawn micrdpipette containing approximately 5 μl of buffer solution (154 mM KCl, 6 mM NaCl, 5 mM MgCl₂, 0.1 μM CaCl₂, 10 mM Hepes) with 10% RNase inhibitor was attached to a motor driven micromanipulator. Once identified, individual cells were drawn into the pipette tip by suction and transferred to an eppendorf tube. Samples were immediately snap frozen on dry ice and stored at −80° C. until enzymatic amplification of total cellular mRNA.

Reverse Transcription Polymerase Chain Reaction (RT-PCR): Cells were thawed in the presence of reverse transcription (RT) reaction components (250 mM Tris-HCl pH 8.3, 375 mM KCl, 15 mM MgCl₂, 2 mM dNTPs, 10 mM DTT) including RNase inhibitor but without Reverse Transcriptase (RT) or DNase I. The reaction mixture was denatured at 65° C. for 1 minute and quickly cooled on ice for 3 minutes. Then RT and DNase I were added and the reaction was allowed to continue at 42° C. for 1 hour. The primer used for the first strand synthesis was oligo-dT including at its 5′ end a recognition sequence for T7 RNA polymerase. This sequence enabled subsequent transcription of antisense RNAs by T7 RNA polymerase. One quarter of the resultant cDNA was amplified by 40 cycles of PCR using primers specific for glyceraldehyde-3-phosphate dehydrogenase (GAPDH) (Clontech) to demonstrate the presence of intact mRNA in the isolated cell.

Antisense RNA amplification: Double stranded cDNA was synthesized from the RT products and amplified by transcribing with T7 RNA polymerase (first round). The RNA generated this way is antisense (aRNA). Random hexamers were used to convert this aRNA into cDNA. After second-strand cDNA synthesis, aRNA was transcribed using radiolabeled nucleotides (second round). In order to determine the size distribution, half of the ³²P-labeled aRNA was analyzed by Northern gel electrophoresis (Sambrook, J. et al., “Molecular Cloning,” 2^{nd} Edn., Cold Spring Harbor Laboratory Press. pp. 7.43-7.45 (1989), which is hereby incorporated by reference).

cDNA clones: cDNA clones were identified in the NCBI database of expressed sequence tag libraries (http://www.ncbi.nlm.nih.gov/dbEST/index.html) using the Genbank accession numbers of the genes. Only those EST clones isolated from exclusively oligo-dT primed libraries were considered. Homology and position of the EST clones were checked against the original sequence of the genes of interest. EST clones were ordered from the companies which held them (ATCC, Genome Systems, Inc., or Research Genetics, Inc.). One microgram of each linearized cDNA was denatured with the addition of 1/10 volume 1 M NaOH and incubated at room temperate for 15 minutes. The sample volume was increased to 100 μl with 10×SSC and the cDNAs of interest were immediately applied to a 96 well format dot blot apparatus (Gibco/BRL) containing a prewetted nylon membrane (MSI).

RNA profiling: Radiolabeled amplified total cellular antisense RNA was used to probe dot blots of cloned cDNAs of interest. Membranes were placed into 15 ml screw cap tubes along with 2.5 ml of hybridization solution, (50% formamide/5×SSPE/5× Denhardt's solution/0.1% SDS/10% dextran sulphate/50 μg/ml denatured salmon sperm DNA/100 μg/ml tRNA). The membranes were prehybridized at 42° C. for a minimum of 4 hours. The cDNA probe was split between two membranes (approximately 1×10⁶ cpm each) to provide duplicate hybridizations. After overnight incubation, blots were washed in 2×SSC/0.1% SDS at 55° C. for 1 hour, 2×SSC/0.1% SDS/10 μg/ml RNase (Ausubel, F. M. et al., “Current Protocols in Molecular Biology.” Supplement 26, John Wiley and Sons Inc., pp. 4.10.4 (1994), which is hereby incorporated by reference) at 37° C. for 1 hour, and 2×SSC/0.1% SDS at 37° C. for 1 hour. Membranes were exposed to a storage phosphor screen for quantitation. Signals were detected by laser densitometric scanning (Phosphorimager Model 425E, Molecular Dynamics). Values (counts) for each cDNA obtained by phosphoimager analysis were manipulated by subtracting an assigned background level, determined by the value obtained by Glial fibrillary acidic protein (GFAP) alone. When the specific values were used for comparison between samples, they were normalized to the total input counts, reflected by the value obtained by pBluescript (pBS). To determine the correlation between the input aRNA and the signal strength, linear regression analyses was performed and the results were expressed as r values for each cDNA tested. For RNA profiling, the average of data from 9 single cells were taken, and the variation within an assay was expressed as standard error of the mean (s.e.m.) and coefficient variance (CV).

### Example 2

### Single Cell Analysis of Tissue from Alzheimer's Disease Patients

Single cell analyses of tissue from Alzheimer's disease patients have been undertaken since neurons appear to be in different stages of the disease even within one microenvironment of AD tissue. The single cell method has the advantage of two molecular techniques, in situ hybridization and aRNA analysis which enable the ability to determine changes in message levels within individual neurons. In situ hybridization provides the ability to analyze a large number of neurons within numerous sections of tissue, and to determine message levels by grain counting. While a powerful tool, one significant drawback of in situ hybridization is the length of time to obtain data for each individual message chosen. To address this issue, the aRNA technique has been developed, to provide the ability to simultaneously test a large number of messages within individual neurons. It is anticipated that the more rapid, broader analysis of many messages at once which is afforded by the aRNA technique will improve the ability to rapidly profile messages within individual neurons of AD. The powerful combination of in situ hybridization and aRNA analysis should lead to a broader overview of the message profiles within single neurons responding to Alzheimer's disease.

To further add to the power of these profiles rather than assess just the difference between AD neurons and age-matched control neurons, neurons will be immunocytochemically classified according to markers of AD which have been developed over the last two decades. Both the in situ technique and the aRNA technique are able to be combined with immunocytochemistry on the same section (or smear), so that an analysis of specific populations of AD neurons is possible. The immunocytochemical markers provide the power to classify neurons into presumed stages of the AD disease, and then to ask specific questions regarding molecular changes within each class of neurons.

The ISH and aRNA methodologies, combined with immunocytochemistry, therefore result in the ability to 1) sort AD neurons immunocytochemically into classes based on current knowledge of the disease progression, 2) quantitatively determine message levels in neurons affected as well as neurons that appear to be unaffected (immunocytochemically defined), and 3) quantitatively determine expression profiles that appear to be changing within a neuron at a presumed stage of the disease.

The combined immunocytochemistry/in situ in hybridization technique was employed to determine within the same section whether neurons containing both a nucleus and frank NFT (a point presumed to be one of the end-stages of the disease for a neuron) exhibited alterations of grain densities for messages related to 1) synaptic integrity (synaptophysin), 2) total message (poly A+), and 3) cell stress (cathepsin D) in single neurons of Alzheimer's disease hippocampus. Synaptic messages were chosen to serve as an indicator of synaptic integrity, and therefore, as a barometer of effective neuronal function.

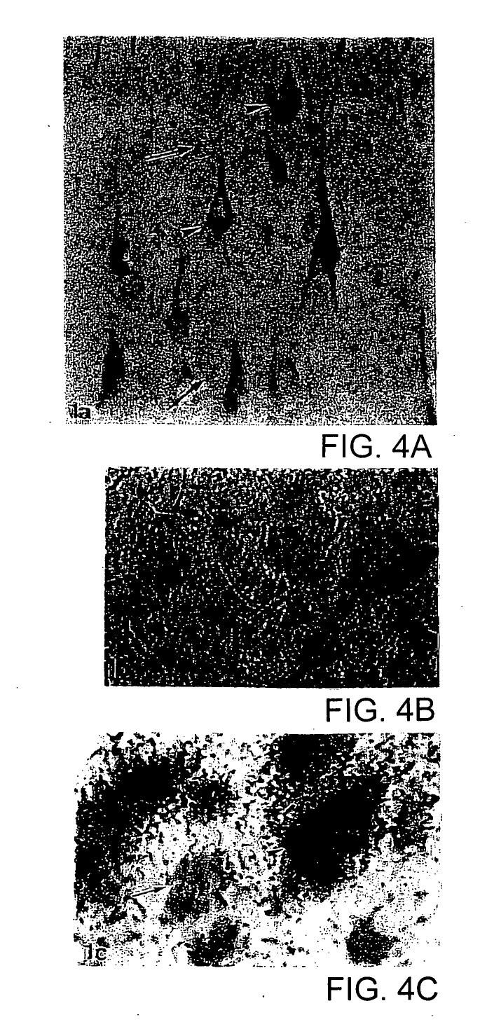

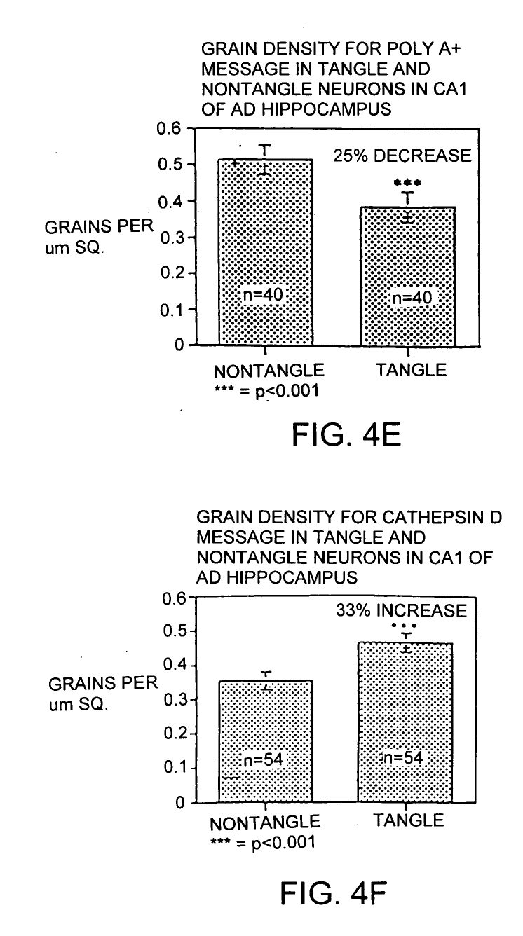

Neurons containing NFT were immunocytochemically localized with mAb 69, which detects a conformational epitope of tangles and combined with in situ hybridization for synapse-related proteins, GAP-43 (Callahan, L. M. et al., “Prelimenary Evidence: Decreased GAP-43 Message in Tangle-Bearing Neurons Relative to Tangle-Free Neurons in Alzheimer's Disease,” Neurobiol. Aging, 15:381-6 (1994), which is hereby incorporated by reference, and synaptophysin, cathepsin D and poly A+ (Callahan, L. et al., “Neurons Bearing Neurofibrillary Tangles Are Responsible for Selected Synaptic Deficits in Alzheimer's Disease.” Neurobiology of Aging, 16:311-314 (1995), and FIG. 4, which is hereby incorporated by-reference). Quantitation of lower grain density sections than that depicted in these figures was performed as a blind analysis with respect to the message type. Synaptophysin grain density (FIG. 4D) was decreased 50% (p<0.0001) in hippocampal CA1 NFT neurons relative to NFT-free neurons (defined as a neuron with a prominent nucleus which did not contain NFT and was within the same 40× field as the tangle neuron). The decrease. in synaptophysin grain density was specific since poly A+ grain density decreased only 25% in NFT neurons relative to NFT-free neurons (FIGS. 4B and E), and cathepsin D grain density, increased 33% in NFT neurons relative to NFT-free neurons (FIG. 4C and F). These data indicate that a dramatic, specific, downregulation of synaptic message is associated with the events related to NFT formation. The study, performed as a blind analysis, examined neurons from 11 AD cases and 7 age-matched controls. Data for synaptophysin grain density of age-matched control neurons compared to grain density in NFT-free neurons in AD brain indicated a 16% decrease in NFT-free neurons. This decrease in synapse message in NFT-free neurons may be due to a decrease of overall synaptic contacts available in AD and/or that populations within the NFT-free category, such as those with phosphorylation of key tau epitopes, show variable degrees of synaptophysin message.

### Example 3

### Dissecting the NFT-Free Neuronal Population

A double immunocytochemistry method combined with in situ hybridization was developed to enable the ability to located neurons with phosphorylated tau epitopes which did not contain NFT. The sections resulting from this technique exhibit the first antigen identified with brown reaction product, the message identified by emulsion grains and the second antigen identified with blue reaction product.

In the first study (FIGS. 5A and B), double immunocytochemistry to identify NFT-free neurons with diffuse phosphorylation of the serine 396 & 404 epitope of tau (recognized by PHF-1) was combined with in situ hybridization for synaptophysin message. The antibodies were chosen since NFT are reliably identified by Mab 69, and PHF-1 is believed to be one of the most sensitive markers of an earlier stage of the AD disease. FIG. 5A demonstrates PHF-1 “only”. neurons in blue (large arrow), neurons containing NFT in brown (arrowhead), and NFT-free neurons showing neither brown nor blue but obvious neuronal nuclei (arrow).

Quantitation of grain counts indicated no significant decrease in synaptophvsin message in neurons diffusely positive for PHF-1 with no NFT relative to NFT-free neighbors (FIG. 5B). These data indicate synaptic message decreases are not seen in neurons that contain tau phosphorylated at the 396/404 epitope.

It is of interest that both the single ICC study (above) and the double ICC study confirm that a roughly 50% decrease of synaptophysin message occurs in NFT neurons in AD. This is of note since the two studies were performed by two different individuals and employed two different counting methods (data for FIG. 4D utilized the MCID image analyzer, data for FIG. 5B was obtained by grain counting using a 100× objective and a manual clicker).

### Example 4

### The Consequence of Phosphorylation of a Tau Epitope

The epitope of serine 262 of tau was chosen since this epitope was demonstrated to be critical for the binding of tau to microtubules (Gustke, N. et al., “Domains of Tau Protein and Interactions With Microtubules,” Biochemistry, 33(32):9511-22 (1994), and Biemat, J. et al., “Phosphorylation of Ser 262 Strongly Reduces Binding of Tau t Microtubules: Distinction Between PHF-Like Immunoreactivity and Microtubule Binding,” Neuron, 11:153-63 (1993), which are hereby incorporated by reference). suggesting phosphorylation of this epitope is one of the events leading to a reduction of microtubules within AD neurons. It was sought to determine if decreased grain density for the synaptic vesicle protein, synaptophysin, was associated with phosphorylated tau serine 262 positive neurons. The study examined 10 AD cases and 4 age-matched controls.

Qualitative observations (FIG. 6) indicated that while variability exists within the population of NFT-free neurons exhibiting phosphorylation of the tau serine-262 epitope (arrow), a number of the phospho-262 positive neurons exhibited a decrease in synaptophysin message grain density relative to ICC negative neighbors (arrowheads). Sections were incubated first with monoclonal antibody mAb 69 to identify tangles (brown). The sections were then processed for in situ hybridization for synaptophysin message (grains). Sections were then processed immunocytochemically with antibody AS32 to the phosphorylated epitope of serine 262 of tau. Quantitation of the 262 data is currently underway.

The ability of immunocytochemically define classes of neurons such as demonstrated in FIGS. 7 through 9 enables the dissection of neuronal populations in AD. By determining profiles of genes within defined populations of neurons, significant information should be gained as to the pathogenesis of AD.

### Example 5

### Isolation of Single Cells

Cell spreads from post-mortem human brain was prepared as described above. The quality of RNA after processing was confirmed by acridine orange staining. Intact RNA exhibits orange fluorescence anid DNA green, thus cytosol is orange and the cell nuclei yellow (FIG. 7A). Acridine orange staining also revealed the part of the slide that would provide optimal cell isolation. This was consistently the area just in from the beginning or head of the spread. As the field of view traveled towards the end of the spread, fewer orange cells and more green cells were seen until at the end only green cells and DNA ‘ghost’ (strands of DNA from cells which had been burst open) were visible.

To identify neurons, IHC on cell spreads was carried out using an anti-neurofilament antibody. NF-H positive neurons are shown in FIG. 7B. Cells of interest were isolated and subjected to reverse transcription. An aliquot of the RT reaction from each single cell was used for PCR in order to establish the presence of intact RNA before proceeding with subsequent steps. 40 cycles of PCR using primers specific to GAPDH, yielded a fragment of 450 bp. This assay served as a screen for RNA positive cells. For example, among 46 cells picked from a control brain, 7 were RT-PCR positive. However two RT-PCR negative cells included in the subsequent procedure also gave rise to aRNA. but the yield was lower.

Fixation methods other than 70% ethanol/150 mM NaCl, including formalin, 4% paraformaldehyde, Carnoys solution have been tried. Acridine orange staining showed that RNA was well preserved by all these methods. However, only cells isolated from tissues fixed in 70% ethanol/150 mM NaCl can be subjected directly to RT-PCR without acid or proteinase K treatment.

### Example 6

### Size Distribution of aRNA Transcripts

The remainder of the RT reaction from each single cell was utilized for RNA amplification and expression profiling. Northern analysis was used to determine the size distribution of the radioactive aRNA used for dot blot hybridization, the aRNA transcripts from all cells ran as a smear ranging from a few hundred bp to 4.4 Kb on a 1.5% denaturing gel (FIG. 8). Since the aRNA library is 3′ enriched (due to transcription beginning at the 3′ end of the gene by the poly A tail), short aRNA transcripts create a need to use extreme 3′ cDNA sequences for dot blotting.

### Example 7

### Feasibility of Dot Blot Analysis

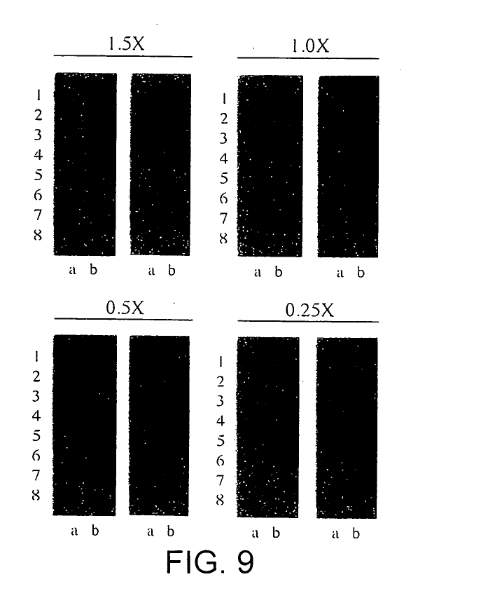

Dot blot analysis used in this study is different from the conventional method. Instead of quantifying the amount of RNA immobilized on the membrane, the relative amount of RNA is measured in the hybridization solution. Similar reverse Northern blot analysis has proven useful for nuclear run-on assays. To verify the methodology, aRNA from a single cell was used at 4 different concentrations for hybridization with several cDNAs (FIG. 9). In order to serve as a quantitation assay, the signals for each cDNA have to be a linear function of the aRNA concentration. Linear regression analyses for each cDNA yielded r values as follows: heat shock protein 70 (HSP70, 0.99), p53 (0.98), HII (0.96), actin (0.96), presenilin 2 (STM2, 0.96), nestin (0.98), cyclin D1 (0.87), calcium/calmodulin-dependent kinase II (CamK II, 0.89), presenilin 1 (S182, 0.97), α-1-antichymotrypsin (α1-Act, 0.98), GAPDH (0.89). Except for cyclin D1, CamK II and GAPDH, the r value for other cDNAs with a broad range of abundance were above 0.96. This study suggests that in this assay dot-blot hybridization is suitable for quantitation of aRNA. It was noted that the pBS gave a higher background than the cDNA for GFAP in pBS. Two other vectors, pGEM3 and pcDNA3 have been tested, and their levels are the same as that of GFAP. The reasons for high background problem associated with pBS alone are unknown. Since the nonspecific signal from pBS was also a linear function of the input counts (r=0.92), it was used for normalization of the specific signals in order to make comparison between cells.

### Example 8

### RNA Profiling

RNA profiling was conducted on 9 neurons from one control brain with 5 cDNAs selected on the basis of their signal strength and also on their potential interest. The cDNAs used were α1-ACT, GAPDH, CamK II, cyclin D1, and nestin. The duplicated blots gave consistent results with standard deviations smaller than 24% (except for one 26% and one 30%). Table 1 shows the average of data points in duplicate for each cDNA, the mean of the data from 9 neurons, the s.e.m. and the CV for each cDNA. Although there is some cell to cell variation as would be expected, the differences in expression of each of the genes examined in nine cells appear to be small as indicated by the CV. Because the blots had been treated with RNase, the remaining counts for individual cDNAs are dependent on the length of the cDNA probes used. They do not represent original abundance of RNAs. However, their relative levels should be consistent from assay to assay.

TABLE 2
The results of RNA profiling on nine single neurons from a control brain with 5 cDNAs.

| CDNA | 1 | 2 | 3 | 4 | 5 | 6 | 7 | 8 | 9 | Mean | s.e.m. | CV |
|---|---|---|---|---|---|---|---|---|---|---|---|---|
| α1-ACT | 6.53 | 3.58 | 4.61 | 5.57 | 5.82 | 6.16 | 5.08 | 4.79 | 6.18 | 4.83 | 0.31 | 5.15 |
| GAPDH | 4.63 | 3.95 | 3.23 | 4.46 | 4.60 | 5.83 | 3.94 | 4.33 | 6.27 | 4.12 | 0.32 | 4.37 |
| CamK II | 3.29 | 3.08 | 2.63 | 3.98 | 2.80 | 4.22 | 3.07 | 2.81 | 4.71 | 3.06 | 0.24 | 4.21 |
| cyclin D1 | 3.59 | 2.55 | 2.84 | 4.38 | 2.92 | 4.72 | 3.31 | 2.95 | 4.84 | 3.21 | 0.29 | 3.69 |
| Nestin | 3.99 | 2.67 | 3.05 | 3.89 | 3.00 | 5.45 | 3.31 | 3.30 | 4.47 | 3.31 | 0.29 | 3.81 |

### Example 9

### Additional Materials And Methods

Tissue Acquisition and Processing. Tissues containing the hippocampal formation were obtained at autopsy from presumptive control and AD brains. All the tissues were well characterized by the Clinical and Neuropathological Cores of the Rochester Alzheimer's Disease Center. The information on the tissues used in this study is summarized in Table 1. In all the AD samples, AD was the primary disease of the patient. The age-matched “control” samples, although clinically nondemented, on subsequent neuropathological examination were assessed at Braak stage I-III (Braak, H. et al., Acta Neuropathol., 82:239-259 (1991), which is hereby incorporated by reference) and were, therefore, classified as early AD for this study. For single-cell aRNA study, fresh tissues were processed immediately at autopsy as described (Cheetham, J. E. et al., J. Neurosci. Methods, 77:43-48 (1997), which is hereby incorporated by reference). In brief, cell layers in the CA1 and subiculum were dissected, trypsinized, smeared gently on microscope slides, fixed in 70% ethanol/i 50 mM NaCl, and stored at −80° C. until use. For ISH, fresh tissues were fixed in 4% paraformaldehyde, cryoprotected in 30% sucrose, and kept frozen at −80° C. until use.

aRNA Profiling. Large pyramidal neurons were isolated from tissue smears under microscopy with micropipettes. To obtain enough material from single cells for gene expression analysis, mRNA from single cells was amplified according to Eberwine et al. Eberwine, J. et al., Proc. Natl. Acad. Sci. U.S.A., 89:3010-3014 (1992), which is hereby incorporated by reference), with modifications. In brief, single cells were treated with DNase I (Life Technologies) followed by reverse transcription with SuperScript II (Life Technologies) according to manufacturer's instructions. The second-strand cDNA was synthesized by a replacement reaction. The double-stranded cDNA was then used as a template for in vitro transcription with T7 RNA polymerase. Because the RNA made in this way is antisense, the procedure is called single-cell antisense RNA (aRNA) amplification. After initial amplification, aRNA served as a template for second-round cDNA synthesis, followed by second aRNA synthesis in the presence of [α⁻³²]CTP (NEN). The radiolabeled aRNA was hydrolyzed in 0.2 M sodium carbonate (pH 10.2) at 60° C. for 40 min and used as a probe for reverse dot-blot hybridization analysis. One microgram of each linearized cDNA was denatured and dot-blotted on a nylon membrane (Micron Separations). For each ³²P-labeled aRNA from a cell, duplicated dot blots were used for each hybridization reaction. Hybridization was performed as described (Cheetham, J. E. et al., J. Neurosci. Methods, 77:43-48 (1997), which is hereby incorporated by reference), and the membranes were exposed to a storage phosphor screen for quantification. Hybridization intensity of each spot was detected by laser densitometric scanning (Phosphorlmager model 425E, Molecular Dynamics). To normalize for the amount of plasmid DNA on each spot, membranes were stripped by incubating in hybridization solution without dextran sulfate at 65° C. for 1 h and reprobed with end-labeled oligonucleotide (TAATACGACTCACTATAGGG) (SEQ. ID. No. 1) specific to the T7 promoter region in plasmid vectors such as pBluescript and pT7T3D.

The National Center for biotechnology Information dbEST database was searched for 3′ cDNAs of interest and purchased them from the distributors (Genome Systems, Genetic Research, or American Type Culture Collection). [The cDNA clones used in this study (with their GenBank accession nos. in parentheses) are as follows: α1-antichymotrypsin (α1-ACT, T40002), glyceraldehyde-3-phosphate dehydrogenase (GAPDH, T71597), heat shock protein 27 (HSP27, T49404), HSP90 (T51115), neurofilament-M (NF-M, T29264), ferritin I. (AA112158), ferritin H (AA111970), ras-like protein (TC25, AA112882), CDK-4 (AA113040), cyclin B1 (AA113937), cyclin G1 (AA114184), cyclin E (T54122), wee1 (T63957), and αB-crystallin (AA192311).] All of the 20 cDNA clones used in this study were sequenced to ensure the identify of cDNAs. it should be noted that if a CDNA has a region that is highly conserved among members of the gene family, cross-reactivity upon hybridization is expected.

RNA Preparation for “Spiking” Experiment. Three λ DNA HindIII fragments of various length (0.5, 2.0, and 6.6 kb) were subcloned into a pSP64 poly(A) vector (Promega). Linearized plasmic DNA was used for in vitro transcription to generate a large quantity of artificial λ poly(A)⁺ RNA. The molar concentration of each λ RNA was determined by spectrophotometry at 260 nm. Total human RNA was isolated from hippocampus of a non-AD sample by using Trizol reagent (Life Technologies) and quantified. Three λ RNAs were mixed at equal amounts, diluted in a 5-fold series (1:5, 1:25, 1:125, and 1:625), and added to total human hippocampal RNA. The spiked and unspiked RNAs were taken through one cycle of amplification as described above. The radiolabeled aRNA was used for reverse dot-blot with λ plasmid DNA. For reprobing the blot, oligonucleotide probe GATTTAGGTGACACTATAG (SEQ. ID. No. 2) specific to the SP6 promoter in pSP64 poly(A) vector was used.

Statistical Analysis. A number of neurons were sampled from both early- and late-stage AD brains. A total of seven cells were analyzed in duplicate from each brain. For each cell, measurements were made on a total of 20 genes. the data were normalized in two stages, first using individual vector measurements and then using the average of each marker across all cells. The resulting standardized data were analyzed statistically to determine differences between early- and late-stage AD brains.

The first level of analysis involved univariate analysis of variance (ANOVA) for each of the 20 markers. In the ANOVA model, cells were nested within brains that were classified as either early- or late-stage AD so that brain were nested within disease state. In this nested model, the comparison of early- and late-stage AD brains is essentially a t test using the brain means and having degrees of freedom equal to the number of brains minus 2, illustrating the importance of including as many brains as possible in the analysis. In addition, the ANOVA provides estimates of variability among replicate measurements, cells, and brains.

Because a large number of markers was included in these analyses, it was also important to describe the overall pattern of difference between early- and late-stage AD brains across all the markers. For this reason, multivariate analysis was also performed by using a canonical discriminant analysis (Kshirsager, A. M., Multivariate Analysis, (1972), which is hereby incorporated by reference). This is essentially a data reduction technique that computes linear combinations (weighted sums) of the original variables to yield a set of canonical variables. The first canonical variable provides the maximal amount of information concerning group differences and subsequent canonical variables are uncorrelated with earlier variables and contain the maximal amount of the remaining information. Unlike principal components analysis, canonical analysis relies on predefined groups. The goal of the analysis is to summarize information on group differences in the data using the canonical variables. The analysis is usually descriptive, and plots of the first two or three canonical variables and of the listing standardized original values contributing most heavily to the canonical variables are examined. Because canonical analysis relies on pre-defined groups, unrelated analyses were done in which either two or five groups were defined. When two groups were delineated, cells were defined as derived from either late- or early-stage AD brain. When five groups were delineated, cells were defined as derived from each of the five brains studied, with no information provided as to disease state. This latter definition is a conservative approach from the point of view of distinguishing brains at different disease stages and should allow a more objective evaluation of differences between early- and late-stage AD brains.

ISH. A partial neurofilament medium subunit (NF-M) cDNA clone (GenBank accession no. T29264) in pT7T3D plasmid vector was purchased (Genetic Research) and sequenced. This clone contains the last 109 bp of the coding region (base pairs 5,442-5,551) and a 227-bp 3′-untranslated region, base pairs 5,442-5,778, based on the sequence published by Myers et al. (Myers, M. W. et al., EMBO J., 6:1617-1626 (1987), which is hereby incorporated by reference). The entire sequence is unique to NF-M, with no sequence homology to neurofilament light or heavy subunit. RNA probes were made in the presence of ³⁵S-labeled UTP by using the Promega Riboprobe system. Methods for ISH were as described (Angerer, L. M. et al., Methods Cell Biol., 35:37-71 (1991), which is hereby incorporated by reference), with modifications for fixed human postmortem tissue (Callahan, L. et al., Neurobiol. Aging, 16:311-314 (1995); Callahan, L. M. et al., Neurobiol. Aging, 15:381-386 (1994), which are hereby incorporated by reference). In brief, 18-μm sections were treated with proteinase K (1 μg/ml) at 37° C. for 30 min and then refixed for adherence with 4% paraformaldehyde for 20 min. After glycine and acetylation treatments, slides were incubated with probes at 56° C. overnight. Slides were then washed extensively in 4×SSC/0.2 M DTT, followed by a high-stringency wash in 50% formamide/0.3 M NaCl/0.02 M Tris-Hcl, pH 8/1 mM EDTA/0.01 MDTT, at 65° C. for 30 min. Slides were then treated with RNase A (20 μg/ml) at 37° C. for 30 min and washed under conditions of increasing stringency (final wash, 0.1×SSC at 50° C.). After air drying, slides were dipped in NTB2 emulsion, exposed for 4 weeks, developed and counterstained with hematoxylin/cosin. By using the captured image of the neuron on a video screen and bright-field illumination, the area of the neuron was traced by a MCID image analyzer and recorded in a computer (MicroVideo Instruments, Ontario, Canada). Grains over cells were counted manually by using a ×100 oil-immersion objective. Fifty neurons in the CA1 and subiculum per section and two sections per brain on separate slides were counted. The grain density for each brain was expressed as an average of counts from 100 neurons plus standard deviation.

### Example 10

### Determination of the Accuracy of Measurement

The previous examples show a method of preparing tissue smears from human postmortem brain and using single whole cell bodies isolated from the smears for RNA profiling based on a single cell approach developed by Eberwine et al. (Eberwine, J. et al., Proc. Natl. Acad. Sci. U.S.A., 89:3010-3014 (1992), which is hereby incorporated by reference) with modifications. The entire procedure can be divided into three parts: (i) isolation of single neurons, (ii) aRNA amplification, and (iii) analysis of expression profiles. The amplified aRNA size ranged from a few hundred base pairs to 4.5 kb and is of good complexity. Reverse dot-blot hybridization was used as a quantitative measurement of RNA levels. In the those experiments (Cheetham, J. E. et al., J. Neurosci. Methods, 77:43-48 (1997), which is hereby incorporated by reference), hybridization intensity was quantitatively related to concentrations of input RNA for three cDNAs with different levels of RNA. However, the linear relationship was less evident for another three cDNAs, which can be ascribed to two different sources: (i) different behavior for different cDNA and (ii) other experimental variations. To distinguish these two possibilities the same experiment was repeated and included 14 more cDNAs. Radiolabeled aRNA from a single cell was used at four concentrations (1.5×, 1.0×, 0.5×, and 0.25×) for hybridization with 20 cDNAs that were selected on the basis of their potential relevance to the pathogenesis of AD. A few control genes were also included in the assay. The cDNAs were categorized as follows: (i) a housekeeping gene. glyceraldehyde-3-phosphate dehydrogenase (GAPDH); (ii) stress genes. HSP27, HSP90. αB-crystallin, ferritin L and H subunits; (iii) cell cycle/apoptosis genes, cyclins D1, B1, G1, and E, CDK4, and wee1; (iv) structural genes, nestin and NF-M; (v) a transcription factor, CREB; (vi) others, presenilin 1, ras-like protein (TC25), α1-ACT, tuberin, and GAD. Some degrees of data variation were noted that may be partly caused by the variation in loading DNA. After normalization of aRNA signal to the plasmid signal on each spot, the specific hybridization signals for each cDNA were plotted against the concentrations of the input aRNA. The r² values for all cDNAs with different levels of RNA abundance were all higher than 0.854, indicating a linear relationship between concentration of aRNA and signals detected. This suggests that dot-blot hybridization can serve as a semiquantitative assay to measure the abundance of multiple RNA species in a single hybridization experiment.

The amount of plasmic DNA on each spot was monitored by reprobing the blot with a probe specific to the vector part of the plasmid and the variability of the same batch of plasmid DNA on separate blots was examined. The study of 70 blots containing a set of 20 cDNAs showed a blot-to-blot variation between 15.4 and 40.8%.

### Example 11

### Amplification Reactions

To demonstrate that the enzymatic reactions in the procedure are linear and to not result in nonproportional amplification of RNA, three artificial λ poly(A)⁺ RNAs were used to spike the total RNA isolated from human hippocampus. Because there is no endogenous λ RNA in total human RNA, the amplified λ RNAs were able to be monitored and to determine whether their levels correlate with the amounts added. Three λ RNAs (0.5, 2.0, and 6.6 kb) were added to human RNA (100 ng/μl) at four concentrations of λ RNA: 20, 4, 0.8, and 0.16 ng/μl, respectively. Hybridization signal for λ RNA was not detected in the unspiked sample. It is evident in FIG. 1A that hybridization intensity is influenced by two factors, the amount of the aRNA probe used and that of plasmid DNA immobilized on the filter membrane. However, when the molar excess of DNA over RNA is great, the amount of DNA beyond that will have little influence on a total amount of hybridization (FIG. 1A, compare 0.5-kb synthetic λ RNA in blots c and d). Normalized hybridization intensity was plotted against the molar concentration of λ RNA added. The specific signal is linearly related to the RNA concentration between 0.2 and 120 nM for all three RNA species (FIG. 1B). The respective r² values for 0.5-, 2.0-, and 6.6-kb λ RNAs are 0.993, 0.947, and 0.988. respectively. The findings are consistent with a recent study by Lockhart et al. (Lockhart, D. J., Nat. Biotechnol., 14:1675-1680 (1996), which is hereby incorporated by reference) who used similar procedures for the reverse-transcription reaction, second-strand cDNA synthesis, and in vitro transcription of aRNA but used a method different from dot-blot hybridization for the detection of signals. FIG. 1B noted that when every RNA was added at equal molar concentration and the corresponding plasmid DNA was spotted at equal molar concentration, the normalized hybridization intensity was directly related to the length of the transcript.

### Example 12

### Gene Expression Profiles

The single-cell approach was used to identify and quantify aRNA for the same 20 genes listed above in multiple cells of each brain in two early- and three late-stage AD brain samples. To compare signals across blots, the individual hybridization intensity of each cDNA on each blot must be normalized to an internal control. There is no known report of any gene whose expression level remains constant under every circumstance. The best control for normalization is the average of the measurements for all cDNAs in an assay. FIG. 2 shows that the average of all the measurements is linear to the amount of radiolabeled aRNA used in the assay (r²=0.992). Among the 20 cDNAs selected, the differences in expression between AD at early and late stages appear to be limited to a small number of genes, and the magnitude of change seems to be relatively small (see below). The average of al! the measurements could, therefore, serve as a good internal control.

The single-cell aRNA data were subjected to an analysis of variance (ANOVA). The changes of RNA level were (i) significant (P<0.05) for α1-ACT, cyclin D1, HSP27, GAD, and wee1; (ii) marginal (0.1<P<0.05) for GAPDH, nestin. αB-crystallin, and TC25; and (iii) not significant for CREB, presenilin I, HSP90, NF-M, tuberin, ferritin L and H, cyclins B1, G1, and E, and CDK4. In late-stage AD, expression level was decreased by 37.7% for cyclin D1, by 28.2% for HSP27, and by 19% for GAD. In contrast, the expression level was increased by 12.2% for wee1 and by 24.3% for α1-Act (FIG. 3).

In addition, the variability was estimated for each of the factors tested in the analysis, namely, replication (each cDNA spotted twice) on a blot, cell-to-cell variation within a brain, brain-to-brain variation, and disease state. The proportion of the total variance accounted for by variation in each of these four factors averaged 62%, 16%, 4.4%. and 17.6%, respectively. In general, replication is the biggest source of variation. Despite the efforts to reduce experimental error by normalization, there are still factors which could not be controlled. Therefore statistical analysis was used to distinguish real differences from background noise. The variation for cells within the same brain is larger than variation for brains within the same group. For genes whose expression levels differ significantly between early- and late-stage AD, the variation at the disease level then becomes larger than the cell-to-cell variance. Because the variation among cells within the same brain is not very high compared with the experimental variation, the numbers of single neurons needed to represent a case of AD may not be high.

By simultaneously considering all of the markers (20 cDNAs) in a multivariate analysis, additional information can be derived about altered levels of message expression of single cells due to individual brain differences and to disease-stage differences. Canonical analysis on data from all 20 markers was used (Kshirsager, A. M., Multivariate Analysis, (1972), which is hereby incorporated by reference) to define weighted Linear combinations of cDNAs that distinguish brains. This analysis, by being sensitive to correlation among genes provides information beyond that provided by univariate analysis. First, data from single neurons from CA1 subiculum of five brains were analyzed by comparing cells from brains at an early and late stage of AD (FIG. 4A). Duplicate blots were obtained for each cell from a specific brain to control for experimental variation; thus, each point in this plot represents one of the duplicated data points from a cell. Canonical variable 1 represents the weighted combination of cDNAs that best distinguished the cells as being from early- or late-stage AD brain. Canonical variable 2 is an analysis of residual variation among cells and clearly does not add greatly to the analysis. Cells of late AD brains can be seen to cluster together. Cells of early AD brains are separated from the cluster formed by cells of late-stage AD brains. The influence of each cDNA on canonical variable 1 is presented graphically (FIG. 4B). The genes contributing heavy weights to canonical variable 1, either positive or negative are not necessarily the genes with significant changes in expression identified by ANOVA. the top five genes with the heaviest weights are CREB, cyclin D1, wee1, NF-M, and crystallin.

Canonical analysis was performed again with the same data without using any information as to disease state but defining which cells came from each of the five brains. In this five-group analysis, cells of early-stage AD brains are separated from the cluster formed by cells oflate-stage AD brains, as well as from each other (FIG. 4C). The influence of each cDNA on canonical 1 in the five-group analysis (FIG. 4D) is slightly different from that in the two-group analysis. The top five genes with the greatest influences are CREB, cyclin D1, NF-M, crystallin, and cyclin G1.

When only the nine markers with statistically marginal to significant ANOVA changes in expression were used for a five-group canonical analysis, separation of the five brains was not as marked as with 20 marker. This suggests that markers other than these nine may provide additional information. In this secondary analysis, the size of the canonical coefficient for each gene correlated better with the probability of its expression being significantly different when examined by ANOVA.

### Example 13

### Validation of aRNA Methodology

Comparison of the levels of NF-M aRNA among two early-and three late-stage AD brains by ANOVA revealed no difference (FIG. 5A). To validate the results seen with the aRNA method, ISH (FIG. 5B) was performed to examine neurons from two early- and three late-stage AD samples; these were not necessarily from the same individuals used for the aRNA analysis (Table 1). Quantification of grain density also showed no change in NF-M message in CA1/subiculum neurons of early- and late-stage AD brains (FIG. 5C). The result of gene expression obtained by the aRNA method was consistent with the results obtained by the well-established ISH analysis.

The results presented in the examples demonstrate that (i) the enzymatic reactions in the aRNA method results in linear amplification of RNAs and (ii) using dot blots to analyze levels of aRNA is feasible. This method has been used to obtain RNA profiles of individual neurons of postmortem human brains. ANOVA was performed to compare the expression levels for individual genes n early- and late-stage AD brains. It was found that the expression levels of cyclin D1, HSP27, and GAD were significantly decreased in late-stage AD samples and that expression of α1-ACT and wee1 were increased (FIG. 3). By using multivariate canonical analysis of the single cell aRNA profiling data, differences were revealed in message expression caused by disease state and brain differences. Canonical analysis sorted differences by disease state in the absence of defining a priori which cells or brains came from which disease state. The genes with significant changes in expression identified by ANOVA were not necessarily the ones contributing heavy weights to canonical variable 1, because in a multivariate analysis, the size of a computed variable depends on interactions among all the markers tested. ANOVA analysis and canonical analysis reinforce each other, with the canonical analysis providing a more interactive presentation. The combination of both types of analyses indicates that the single-cell aRNA method is capable of revealing differences in message expression caused by brain differences and disease state.

How changes in expression of certain genes relate to the pathogenesis of AD awaits further study. Expression of many cell cycle regulators in postmitotic neurons has been reported, but the functions of the proteins encoded by these genes mav be unrelated to cell division. Cyclin D1, for example, which is essential for progression through the G₁ phase of the cell cycle, could be induced in postmitotic neurons undergoing apoptosis (Freeman, R. S., Neuron, 12:343-355 (1994); Kranenburg, O. et al., EMBO J., 15:46-54 (1996), which are hereby incorporated by reference). By using the single-cell aRNA approach, a significant 37.7% decrease of cyclin D1 RNA was found in AD. Cyclin D1 usually forms a complex with CDK4, but no change in the expression level of CDK4 gene was detected. Immunoreactivities of cdc2/cyclin B1 were found to be elevated in neurofibrillary tangle-containing neurons in AD and cdc2/cyclin B1 was shown to phosphorylate neurofibrillary tangle and recombinant tau in vitro (Vincent, I. et al., J. Neurosci., 17:3588-3598 (1997), which is hereby incorporated by reference). No change was detected in the expression of cyclin B1 gene in late-stage AD compared with early-stage AD.

Small stress proteins including HSP27 and αB-crystallin have been reported to act as cellular inhibitors of apoptosis (Mehlen, P. et al., J. Biol. Chem., 271:16510-16514 (1996); Huot, J. et al., Cancer Res., 56:273-279 (1996), which are hereby incorporated by reference). A 28.2% decrease of HSP27 RNA was found in AD. it is possible that selective reduction of HSP27 expression in AD may contribute to the vulnerability of certain populations of neurons to neurodegeneration.

Inflammatory mechanisms have been shown to be part of the pathogenic events of AD (Rogers, J. et al., Neurobiol. Aging, 17:681-686 (1996), which is hereby incorporated by reference). In this study, the finding of an elevated level of α1-ACT, an acute-phase protein, in AD is consistent with this hypothesis.

To validate the results seen with the aRNA method, ISH was performed with a marker gene, NF-M. In adult neurons, neurofilaments are important for the maintenance of the highly polarized morphology of axons and for normal axonal caliber. The expression level of NF-M detected by the aRNA method was found to be the same in AD at early and late stages. The result obtained by the wee1 -established ISH analysis was consistent with the aRNA result by using Northern blot and dot-blot analyses, studies have shown a decrease in gene expression for neurofilament light subunit (Lukiw, W. J., Int. J. Neurosci, 55:81-88 (1990); Robinson, C. A. et al., Neurobiol. Aging. 15:681-690 (1994), which are hereby incorporated by reference) and NF-M (Kittur, S. et al., J. Geriatr. Psychiatry Neurol., 7:153-158 (1994) which is hereby incorporated by reference) in AD neocortex. However, in contrast to the reported decrease of NF-M gene expression in AD neocortex, a change in NF-M message was not detected in single neurons of CA1/subiculum in early- relative to late-stage AD brain by two methods. The decrease in NF-M message in AD detected by the “grind and find” type of assay could be due to the loss of neurons in AD brains. The results clearly demonstrate that molecular analysis at the single-cell level should provide further insights into the molecular alterations, either causative or consequential, in AD pathogenesis.

aRNA profiling for a larger number is also possible. Recent advances in obtaining the complete sequence of the human genome and the development of improved technologies for the simultaneous evaluation of the expression of large numbers of genes provide ways to carry out research in human disease. For example, two techniques for analyzing differences in gene expression in normal and cancer cells have recently been reported: sequencing-based serial analysis of gene expression or SAGE (Velculescu, V. E. et al., Science, 270:484-487 (1995), which is hereby incorporated by reference) and high-density cDNA microarray on a glass microscope slide (Schena, M. et al., Science, 270:467-470 (1995), which is hereby incorporated by reference). SAGE has been used in the study of gastro-intestinal tumors to analyze differential expression of 45,000 different genes (Zhang, L. et al., Science, 276:1268-1272 (1997), which is hereby incorporated by reference) and cDNA microarray used in the study of melanoma to examine 870 genes (DeRisi, J. et al., Nat. Genet., 14:457-460 (1996), which is hereby incorporated by reference). Other materials, such as high-density cDNA array on a nylon membrane containing 588 human genes (CLONTECH), array of 18,394 genes (Genome Systems, St. Louis) and high-density oligonucleotide array containing 10,000 genes (Affymetrix, Santa Clara, Calif.) are currently or soon will be commercially available. The combination of aRNA amplification on defined cell populations and high density cDNA or oligonucleotide arrays promises the development of information necessary to fully understand the complexity of the molecular processes in AD and other diseases.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of profiling mRNA production during stages comprising:
isolating a plurality of cells each characterized by a different stage of disease progression;
antisense RNA transcripts from mRNA of each of the plurality of cells;
amplifying the antisense RNA transcripts; and
quantitating the levels of individual antisense RNA transcripts which is indicative of levels of an mRNA for each of the plurality of cells.

2-56. (canceled)
